SEQUENCE LISTING (3 sequences)

SEQ ID NO: 1 - protein, 140 aa, Homo sapiens
MGPRRRSRKPEAPRRRSPSPTPTPGPSRRGPSLGASSHQHSRRRQGWLKEIRKLQKSTHLLIRKLPFSRLAREICVKFTRGVDFNWQAQALLALQEAAEAFLVHLFEDAYLLTLHAGRVTLFPKDVQLARRIRGLEEGLG

SEQ ID NO: 2 - protein, 114 aa, Homo sapiens
MGPRRRSRKPEAPRRRSPSPTPTPGPSRRGPSLGASSHQHSRRRQGWLKEIRKLQKSTHLLIRKLPFSRLAAEAFLVHLFEDAYLLTLHAGRVTLFPKDVQLARRIRGLEEGLG

SEQ ID NO: 3 - protein, 17 aa, Artificial Sequence
  Synthetic construct CENP-A fragment fromm aa 3 to aa 19
PRRRSRKPEAPRRRSPS

(19) United States
(12) Patent Application Publication
(10) Pub. No.: US 2024/0418722 A1
(43) Pub. Date: Dec. 19, 2024

(54) METHODS AND KITS FOR DIAGNOSING CANCER AND PREDICTING RESPONSE TO TREATMENT BASED ON CENP-A LABELLING

(71) Applicants: INSTITUT CURIE, PARIS (FR); CENTRE NATIONAL DE LA RECHERCHE SCIENTIFIQUE, PARIS (FR); UNIVERSITE CLERMONT AUVERGNE, CLERMOND-FERRAND (FR); SORBONNE UNIVERSITE, PARIS (FR)

(72) Inventors: Geneviève ALMOUZNI, NEUILLY SUR SEINE (FR); Pierre VERRELLE, PARIS (FR); Didier MESEURE, PARIS (FR); Jean-Pierre QUIVY, VINCENNES (FR); Frédérique BERGER, PARIS (FR)

(21) Appl. No.: 18/554,161

(22) PCT Filed: Apr. 6, 2022
(86) PCT No.: PCT/EP2022/059165

§ 371 Date: Oct. 5, 2023

(30) Foreign Application Priority Data
Apr. 6, 2021 (EP) 21305439.8

(51) Int. Cl. G01N 33/574; G01N 33/50; G01N 33/68

(52) U.S. Cl. CPC G01N 33/574; G01N 33/5091; G01N 33/6854; G01N 33/6875; G01N 2800/52

(57) ABSTRACT

Cell fusion techniques have been used to produce hybrids between myeloma cells and antibody-producing cells. The hybrid lines derived are permanently adapted to grow in tissue culture and are capable of inducing antibody-producing tumors in mice.

Spleens from mice immunized against sheep red blood cells (SRBC) were fused to an 8-azaguanine-resistant clone (X63-Ag8) of MOPC 21 myeloma. Over 50% of the derived hybrid lines produce and secrete immunoglobulins different from the MOPC 21 myeloma. About 10% of the hybrid lines exhibit anti-SRBC activity. The high proportion of antibody-producing hybrids suggests that the fusion involves a restricted fraction of the spleen cell population, probably cells committed to antibody production.

In order to avoid the presence of the MOPC 21 heavy chain in the specific hybrids, another myeloma cell line (NSI/Ag4-1) has been used. This is a nonsecreting variant of the MOPC 21 myeloma which does not express heavy chains.

Three anti-SRBC (probably of the μ, γ_{2b} and γ₁ classes, respectively) and two anti-2,4,6-trinitrophenyl (of the μ class antibody-producing hybrids have been repeatedly cloned. By random selection and by selection of specific clones according to their lytic activity (clone plaque selection), a number of different lines have been constructed. Such lines express different combinations of the four possible chains of each hybrid line: the myeloma γ and k chains and the specific antibody heavy and light chains. In three cases (Sp1, Sp2 and Sp7) it is shown that only the specific H and L combination has activity and that the myeloma chains are unable to substitute for them. In most cases lines have been derived which no longer express the MOPC 21 chains but only the specific antibody chains.

### FIELD OF THE INVENTION

Invention belongs to the field of medicine, more specifically the field of cancer prognostic and therapeutic management. Invention relates to an in vitro method of identifying an oncogenic transformation in a tissue sample from a subject suspected to be afflicted with a cancer.

### BACKGROUND OF THE INVENTION

Cancer is the second leading cause of death globally and is responsible for an estimated 9.6 million deaths in 2018. Globally, about 1 in 6 deaths is due to cancer (World Health Organization, <website: who.int>). In Europe, cancer incidence is continuously growing and increased by around 50 percent from 2.1 million to 3.1 million cases between 1995 and 2018 (Hofmarcher et al., 2019).

Accordingly, the economic impact of cancer is significant and is continuously increasing. Direct costs of cancer doubled from €52 billion to €103 billion in Europe between 1995 and 2018 (Hofmarcher et al., 2019), to which should be added significant economic burden of productivity losses due to premature deaths.

From the patient point of view, side effects of treatments, some of which being debilitating, the great variability in terms of malignancy and life duration expectancy make having simple and a clear picture on how the disease will evolve and on treatments options is an issue for personal decision-making.

Further, avoiding use of inappropriate or ineffective therapy is both of interest from the patient point of view and at the level of health policy for providing a more efficient care management and optimizing the use of resources.

In the current era of precision medicine for cancer treatment, there is a growing need for biomarkers enabling to select those patients that will benefit from a given treatment. Mahlke et al. (2020) summarizes the state of the art about CENP-A-chromatin in health and cancer. WO2009/114836 relates to gene sets which are thought to be useful in assessing prognosis and/or predicting the response of cancer, e.g. colorectal cancer to chemotherapy. WO 2017/083675 reports improved Molecular Grade Index (MGI) and Breast Cancer Index (BCI) to obtain more accurate predictions of risk of recurrence and response to treatments of breast cancer. Sun et al. (2016) reports elevated CENP-A expression in a variety of human cancers.

### SUMMARY OF THE INVENTION

Inventors have discovered that CENP-A labelling of tissue sample provides valuable information for diagnosis of neoplastic lesions. More particularly inventors have been able to determine that nuclear pattern of CENP-A labelling is indicative of the presence of an oncogenic transformation in the tested tissue sample.

Also, it is a first object of the invention to provide an in vitro method for identifying an oncogenic transformation in a tissue sample from a subject suspected to be afflicted with a cancer, said method comprising:
a step of labelling said tissue sample for CENP-A protein or for an homolog thereof,
a step of determining a nuclear pattern of CENP-A labelling in cells from said tissue sample.

Indeed, said nuclear pattern of CENP-A labelling is found significantly altered in tissues sample within which an oncogenic transgenic transformation occurred. In that regard, in a particular embodiment of an in vitro method for identifying an oncogenic transformation, the step of determining the nuclear pattern of the CENP-A labelling comprises determining a presence and a subnuclear distribution of CENP-A foci in the nucleus of the labelled cells from said tissue sample. Accordingly, in an embodiment, the present invention relates to an in vitro method for identifying an oncogenic transformation in a tissue sample from a subject suspected to be afflicted with a cancer, comprising:
a step of labelling said tissue sample for CENP-A protein or for an homolog thereof,
a step of determining a nuclear pattern of CENP-A labelling in cells from said tissue sample,
said step comprising determining a presence and a subnuclear distribution of CENP-A foci in the nucleus of the labelled cells from said tissue sample.

CENP-A is found to cluster into foci, inside but specifically at the periphery of the nucleus, and optionally at the periphery of the nucleolus in cells free of oncogenic transformation. Further, specific features of said nuclear pattern of CENP-A labelling have been shown by the inventors as of particular interest, as e.g., the mean number of CENP-foci, the size or the shape of CENP-A foci, and/or more globally, the homogeneity of said labelling. Accordingly, in an embodiment of said method, determining a nuclear pattern of CENP-A labelling further comprises determining, in said tissue sample:
the mean number of CENP-A foci per nucleus,
the size and/or shape of CENP-A foci, and/or
the intra-cell or intra-tissue heterogeneity of CENP-A labelling.

Also, in an embodiment of said method, when the step of determining a nuclear pattern of CENP-A comprises detecting intranuclear CENP-A foci which are distributed only at the nuclear periphery, and optionally nucleolar periphery, said pattern is indicative that no oncogenic transformation occurred in said sample and that tissue sample does not display a malignant lesion. In a more particular embodiment, in that case, from 9 to 18 CENP-A foci are detected in the nucleus at the nuclear periphery and optionally nucleolar periphery in said tissue sample.

Conversely, in another embodiment of said method, when the step of determining a nuclear pattern of CENP-A comprises:
detecting no intranuclear CENP-A foci, or
detecting intranuclear CENP-A foci, at least a part thereof being not distributed at the nuclear periphery or at the nucleolar periphery,
said pattern is indicative that an oncogenic transformation occurred in the sample and that sample tissue is at risk to display a malignant lesion. More particularly, in said embodiment, a mean number of less than 9 or more than 18 CENP-A foci in nuclei of cells is detected in the tissue. More particularly, in said embodiment, the step of determining a nuclear pattern of CENP-A comprises determining at least a part of CENP-A foci are less than 0.6 μm, less than 0.5 μm even more preferably less than 0.4 μm, in at least one of their dimensions.

Inventors have further discovered that nuclear pattern of CENP-A labelling constitutes a valuable prognostic marker of the response of a malignant lesion to radiotherapy, chemotherapy and/or concurrent chemoradiation therapy and is indicative of the Overall Survival of the subject afflicted by the cancer and treated with radiotherapy, chemotherapy and/or concurrent chemoradiation therapy. Nuclear pattern of CENP-A labelling is therefore an invaluable support in term of disease management for treating cancer.

Also a more specific object of the invention relates to an in vitro method for identifying an oncogenic transformation in a tissue sample from a subject suspected to be afflicted with a cancer comprising of labelling said tissue sample for CENP-A protein or for an homolog thereof, and determining a nuclear pattern of CENP-A labelling in cells from said tissue sample, wherein:
detecting intranuclear CENP-A foci, at least a part thereof being not distributed at the nuclear periphery or at the nucleolar periphery, and
detecting an homogeneous intra-cell CENP-A labelling in term of size, number, and/or labelling intensity of CENP-A foci, and
detecting an homogeneous intra-tissue CENP-A labelling in term of size, number, and/or labelling intensity of CENP-A foci,
which is indicative that sample tissue originates from a malignant lesion which is responsive to radiotherapy, chemotherapy and/or concurrent chemoradiation therapy.

Conversely, in another particular object, the invention relates to an in vitro method for identifying an oncogenic transformation in a tissue sample from a subject suspected to be afflicted with a cancer comprising of labelling said tissue sample for CENP-A protein or for an homolog thereof, and determining a nuclear pattern of CENP-A labelling in cells from said tissue sample, wherein: intranuclear CENP-A foci are detected, at least a part thereof being not distributed at the nuclear periphery or at the nucleolar periphery, and wherein said CENP-A labelling of tissue sample is also characterized by an intra-cell or an inter-cell heterogeneity in the size, shape, or number of said foci, said pattern being indicative that tissue sample originates from a malignant lesion which is at risk of being not responsive to radiotherapy, chemotherapy and/or concurrent chemoradiation therapy.

In an embodiment of the methods according to the invention, labelling of the tissue sample for CENP-A protein or for an homolog thereof CENP-A labelling is performed using an antibody, or a fragment thereof, preferably a monoclonal antibody, or a fragment thereof, a recombinant antibody or a fragment thereof, a nanobody or a fragment thereof, or an aptamer, directed against CENP-A.

Chromogenic immunohistochemical methods are particularly suitable to implement the methods of the invention. Also, in an embodiment of any of the methods of the invention, labelling said tissue sample for CENP-A protein or for an homolog thereof is performed using an chromogenic immunohistochemical methods. Accordingly, the method according to the invention is a chromogenic immunohistochemistry method. In that context, inventors have found that using fixative solution comprising less than 1% formaldehyde provide a CENP-A nuclear labelling of a particular quality and interest for implementing the methods of the invention and detecting CENP-A foci. The quality of labelling is even higher when using an Alcohol-formalin-Acetic acid mix. Also accordingly, in a particular embodiment, when the method of the invention is a chromogenic immunohistochemistry method, said method comprises a step of fixing cells of tissue sample with a fixative containing less than 1% formaldehyde or even no formaldehyde, more preferably with an Alcohol-formalin-Acetic acid mix.

In an embodiment, the tissue sample labelled in the methods of the invention the tissue sample have been obtained from a biopsy, a fine-needle aspiration, a core biopsy, or subtotal removal of single node, or even the tumour tissue.

In a preferred embodiment of the methods of the invention, the subject is a mammal, preferably a human subject.

### LEGEND OF DRAWINGS

FIG. 1: CENP-A staining by IHC (Immunohistochemistry) in normal human tissues. Images of CENP-A staining by IHC in the indicated human tissues. Scale bar is 10 μm.

FIG. 2: CENP-A staining by IHC in human carcinomas. Images of CENP-A staining by IHC in non-Hodgkin lymphoma and carcinomas as indicated. Scale bar is 10 μm.

FIG. 3: CENP-A localization patterns as a function of the stage of breast lesions. A) CENP-A staining by IHC in breast tissues as indicated. Invasive ductal carcinoma displaying CENP-A foci localized only inside the nuclear space (left) and few remaining at the nuclear periphery (right) are shown. Scale bar is 10 μm. B) scheme depicting the CENP-A patterns.

FIG. 4: CENP-A localization in SCC61 and SQ20B grafted cells. Upper Panel: Curve diagrams showing normalized average tumor volume from grafted cells as a function of time following 5×4 Gy irradiation, (solid black line) or no irradiation control (solid gray line) for the radio sensitive cell line SCC61 (A; n=8 mice) and the radio resistant cell line SQ20B (B; n=8 mice). The dashed lines represent the 95% confidence interval. Down panel: merge images of z projection with maximum intensity from 3D acquisition of immunofluorescence from 3 representative nuclei with CENP-A staining and DAPI labelling of SCC61 (A) and SQ20B (B) grafted cells. Scale bar is 10 μm.

FIG. 5: CENP-A nuclear localization pattern is a marker of disease control by concurrent chemoradiation therapy (CCRT) in head and neck squamous cell carcinoma (HNSCC) patients. A) CENP-A IHC images of HNSCC biopsies corresponding to CENP-A pattern-C (signature for responsive cancers). Scale bar is 10 μm. A scheme depicting the pattern is shown on the right B) as in A) but CENP-A patterns-non-C. C) Graph plot showing proportion (in %) of disease control (black: no disease control; grey: disease control) in biopsies from patients displaying CENP-A pattern C or non-C. D) Kaplan Meier survival curve of patients treated by CCRT with CENP-A pattern-C and CENP-A patterns-non-C.

### DETAILED DESCRIPTION OF THE INVENTION

### Definitions

As intended herein, the term “comprising” has the meaning of “including” or “containing”, which means that when an object “comprises” one or several elements, other elements than those mentioned may also be included in said object. In contrast, when an object is said to “consist of” one or several elements, the object cannot include other elements than those mentioned.

The expression “oncogenic transformation” refers herein to the multistage process of successive acquisition of genetic and epigenetic alterations affecting cell proliferation and survival (Unni et al., 2008) that leads ultimately to transformation of a normal cell into a cancer cell. Also, identifying an oncogenic transformation comprises determining whether in at least a part of the tested tissue sample, transformed cancer cells are encountered showing that tissue sample displays a cancerous lesion.

The term “subject” is meant to refer to any mammal, e.g., mouse, rat, monkey, dog, human. In a preferred embodiment the subject is a human subject. In another preferred embodiment for implementation of the methods of the invention, subject refers to subject displaying a majority of metacentric chromosomes.

“Tissue sample” includes any sample from e.g., a biopsy, a fine-needle aspiration, a core biopsy, or subtotal removal of single node, tumour resection or even the tumour tissue itself.

“Cancer”, or “malignant neoplasm” or “malignant tumour” or “malignant lesion” refers to diseases where abnormal cells grow uncontrollably, go beyond their usual boundaries to invade adjoining parts of the body and/or spread to other organs. In a more particular embodiment “cancer” is selected from all types of squamous cell carcinomas and adenocarcinomas from different tissues (skin, cervix, head and neck, anus, breast, colorectum, oesophagus bronchus); and also, in a particular embodiment, cancer is selected from brain malignant tumours, testicular cancers, soft tissue and bone sarcomas (angiosarcoma, fibrosarcoma, rhabdomyosarcoma, liposarcoma); lung: bronchogenic carcinomas (squamous cell, undifferentiated small cell, undifferentiated large cell, adenocarcinomas), alveolar (bronchiolar) carcinomas, sarcomas, lymphomas, malignant mesotheliomas, gastrointestinal carcinomas: esophagus (squamous cell carcinomas, adenocarcinomas, leiomyosarcomas, lymphomas), stomach (carcinomas, lymphomas, leiomyosarcomas), pancreas (ductal adenocarcinomas, neuroendocrine carcinomas, carcinoid tumors), small bowel (adenocarcinomas, lymphomas, carcinoid tumors, Kaposi's sarcoma, leiomyoma, hemangioma, lipoma, neurofibroma, fibroma), large bowel (adenocarcinoma, tubular adenoma, Villous adenoma, hamartoma, leiomyoma); Genitourinary tract cancer: kidney (adenocarcinoma, Wilms' tumor nephroblastoma, lymphoma, leukemia), bladder and urethra (Squamous cell carcinomas, transitional cell carcinomas, adenocarcinomas), prostate (adenocarcinomas, Sarcomas), testis (seminoma, teratoma, embryonal carcinoma, teratocarcinoma, choriocarcinoma, Sarcoma, interstitial cell carcinomas); Liver cancer: hepatoma (hepatocellular carcinomas), cholangiocarcinomas, hepatoblastomomas, angiosarcomas; Bone cancer: osteogenic sarcoma (osteosarcoma), fibrosarcoma, malignant fibrous histiocytoma, chondrosarcoma, Ewing's sarcoma, malignant lymphoma (reticulum cell Sarcoma), multiple myeloma, malignant giant cell tumor, Nervous System cancer: brain (astrocytoma, medulloblastoma, glioma, ependymoma, germinomapinealoma, glioblastoma multiform, oligodendroglioma, retinoblastoma), Spinal cord (glioma, Sarcoma); Gynecological cancer: uterus (endometrial carcinomas), cervix (cervical carcinomas), ovaries (ovarian carcinomas, Serous cystadenocarcinomas, mucinous cystadenocarcinomas, unclassified carcinomas, dysgerminomas, malignant teratomas), Vulva (Squamous cell carcinomas, adenocarcinomas, fibrosarcomas, melanomas), vagina (clear cell carcinomas, Squamous cell carcinomas, botryoid Sarcomas, embryonal rhabdomyosarcomas, fallopian tubes (carcinomas); skin cancer: malignant melanoma, basal cell carcinomas, Squamous cell carcinomas, Karposi's Sarcomas, Adrenal glands cancers: neuroblastomas, cavernous, cholangiocarcinoma, chondosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epithelioid, Ewing's sarcoma, fibrolamellar carcinomas, superficial spreading melanoma, undifferentiated carcinomas, uveal melanomas, verrucous carcinoma, well differentiated carcinoma. In another particular embodiment, cancer is selected from brain malignant tumours, testicular cancers, soft tissue and bone sarcomas (angiosarcoma, fibrosarcoma, rhabdomyosarcoma, liposarcoma); lung: bronchogenic carcinomas (squamous cell, undifferentiated small cell, undifferentiated large cell, adenocarcinomas), alveolar (bronchiolar) carcinomas, sarcomas, lymphomas, malignant mesotheliomas, gastrointestinal carcinomas: esophagus (squamous cell carcinomas, adenocarcinomas, leiomyosarcomas, lymphomas), stomach (carcinomas, lymphomas, leiomyosarcomas), pancreas (ductal adenocarcinomas, neuroendocrine carcinomas, carcinoid tumors), small bowel (adenocarcinomas, lymphomas, carcinoid tumors, Kaposi's sarcoma), large bowel (adenocarcinoma, tubular adenoma, Villous adenoma, hamartoma, leiomyoma); Genitourinary tract cancer: kidney (adenocarcinoma, Wilms' tumor nephroblastoma, lymphoma, leukemia), bladder and urethra (Squamous cell carcinomas, transitional cell carcinomas, adenocarcinomas), prostate (adenocarcinomas, Sarcomas), testis (seminoma, teratoma, embryonal carcinoma, teratocarcinoma, choriocarcinoma, Sarcoma, interstitial cell carcinomas); Liver cancer: hepatoma (hepatocellular carcinomas), cholangiocarcinomas, hepatoblastomomas, angiosarcomas; Bone cancer: osteogenic sarcoma (osteosarcoma), fibrosarcoma, malignant fibrous histiocytoma, chondrosarcoma, Ewing's sarcoma, malignant lymphoma (reticulum cell Sarcoma), multiple myeloma, malignant giant cell tumor, Nervous System cancer: brain (astrocytoma, medulloblastoma, glioma, ependymoma, germinomapinealoma, glioblastoma multiform, oligodendroglioma, retinoblastoma), Spinal cord (glioma, Sarcoma); Gynecological cancer: uterus (endometrial carcinomas), cervix (cervical carcinomas), ovaries (ovarian carcinomas, Serous cystadenocarcinomas, mucinous cystadenocarcinomas, unclassified carcinomas, dysgerminomas, malignant teratomas), Vulva (Squamous cell carcinomas, adenocarcinomas, fibrosarcomas, melanomas), vagina (clear cell carcinomas, Squamous cell carcinomas, botryoid Sarcomas, embryonal rhabdomyosarcomas, fallopian tubes (carcinomas); skin cancer: malignant melanoma, basal cell carcinomas, Squamous cell carcinomas, Karposi's Sarcomas, Adrenal glands cancers: neuroblastomas, cavernous, cholangiocarcinoma, chondosarcoma, clear cell carcinoma, cystadenoma, endodermal sinus tumor, endometrial hyperplasia, endometrial stromal sarcoma, endometrioid adenocarcinoma, ependymal, epithelioid, Ewing's sarcoma, fibrolamellar carcinomas, superficial spreading melanoma, undifferentiated carcinomas, uveal melanomas, verrucous carcinoma, well differentiated carcinoma. In a preferred embodiment, “cancer” is selected from head and neck cancer, breast cancer such as and triple negative breast cancer, ductal carcinoma in situ and invasive ductal carcinoma, prostate cancer, colorectal cancer, pancreas cancer, ovary cancer, glioblastoma, lung cancer, non-small cell lung cancer, ovarian cancer, bladder cancer, and cervical cancer. In a more preferred embodiment “cancer” refers to head and neck cancer.

“CENP-A” is a protein which, in humans, is encoded by the CENP-A gene (also known as CENPA, CenH3, centromere protein A, Histone H3-Like Centromeric Protein A, Centromere Autoantigen A); Human CENP-A protein sequences are accessible under the Uniprot number P49450. Two isoforms amino acids sequences for human CENP-A of respectively 140 amino acids (SEQ ID NO: 1, CENP-A isoform 1) and 114 amino acids (SEQ ID NO: 2, CENP-A isoform 2) are described:

CENP-A isoform 1:
(SEQ ID NO: 1)
MGPRRRSRKPEAPRRRSPSPTPTPGPSRRGPSLGASSHQHSRRRQGWLKE
IRKLQKSTHLLIRKLPFSRLAREICVKFTRGVDFNWQAQALLALQEAAEA
FLVHLFEDAYLLTLHAGRVTLFPKDVQLARRIRGLEEGLG.
CENP-A isoform 2:
(SEQ ID NO: 2)
MGPRRRSRKPEAPRRRSPSPTPTPGPSRRGPSLGASSHQHSRRRQGWLKE
IRKLQKSTHLLIRKLPFSRLAAEAFLVHLFEDAYLLTLHAGRVTLFPKDV
QLARRIRGLEEGLG.

A “CENP-A protein or homolog thereof” is a protein whose sequence shares at least 80% homology with the CENP-A protein of SEQ ID NO: 1 or of SEQ ID NO: 2. Preferably, the amino acid sequences of the homologs of the CENP-A protein are identical at more than 80%, preferably 81%, more preferably 82%, more preferably 83%, more preferably 84%, more preferably 85%, preferably 86%, more preferably 87%, more preferably 88%, more preferably 89%, more preferably 90%, more preferably 91%, more preferably 92%, more preferably 93%, more preferably 94%, more preferably 95%, more preferably 96% to the and even more preferably 97% to SEQ ID NO: 1 or SEQ ID NO: 2. In a particular embodiment “CENP-A protein” refers to a protein of SEQ ID NO: 1 or SEQ ID NO: 2.

“Nuclear pattern” refers to the reactivity or pattern that is observed, in response to the exposure to a labelling agent (upon either direct or indirect detection) by any means known in the art like e.g. confocal microscopy or fluorescent immunolabelling or chromogenic immunohistochemistry (i.e. chromogenic immunodetection of the antigen on tissue section), in the nucleoplasm and the nuclear subcomponents (e.g. centromere or nucleolar). “Nuclear pattern” comprises any homogeneous, speckled, centromeric, discrete nuclear dots or foci, nucleolar, nuclear envelope labelling. As described herein, inventors have identified pleomorphic nuclear patterns which are linked to oncogenic transformation of a tissue sample, malignancy and/or responsiveness of a cancerous lesion to cancer treatments. Also, the CENP-A nuclear pattern corresponds to the labelling that is observed when using CENP-A specific detection means, as e.g., antibodies or aptamers. “Nuclear periphery” when related to intranuclear CENP-A foci, refers to the localization of said foci just below nuclear envelope and tagging the shape of the nuclear envelope.

In the field of cancer diagnosis and/or prognosis by immunochemistry, the homogeneity or heterogeneity of the distribution, intensity and features of the labelling within the tumor are criteria that can be considered by anatomical pathologists. “Homogenous” when related to the CENP-A nuclear pattern of a tissue sample, refers to similarity of said pattern all through the tissue sample. In other word a CNEP-A nuclear pattern will be considered as homogeneous when at least 80%, preferably at least 90%, more preferably at least 95% even more preferably 100% display the same CENP-A labelling in terms of number of CENP-A foci per nucleus, localization of CENP-A foci, size and/or shape of foci. Also, a tissue sample will be considered as homogeneous if, within a single nucleus, at least 80%, preferably at least 90%, more preferably at least 95%, even more preferably 100% CENP-A foci display the same size and/or shape. Conversely, CENP-A nuclear pattern will be considered as “heterogenous” when less than 80% of the cells, less than 70%, less than 60% even more preferably less than 50% of the cells display the same CENP-A labelling in terms of number of CENP-A foci per nucleus, localization of CENP-A foci, size and/or shape of foci. Also, a tissue sample will be considered as heterogeneous if, within a single nucleus size, less than 80% of CENP-A foci display the same size and/or shape.

“Chemotherapy” or “chemotherapeutic treatment” as used herein refers to agents (e.g. cytostatic or antineoplastic) which are used against cancer and destroy cancer cells by non-specific mechanisms as inhibition of mitosis or DNA damage.

“Radiotherapy” or “radiotherapeutic treatment” is a therapy using ionizing radiation, generally as part of cancer treatment to control or kill malignant cells. Radiation therapy may be curative for cancer localized to one area of the body. Radiotherapy can also be used as an adjuvant therapy, to prevent tumor recurrence after removal of the primary malignant tumor. Radiotherapy can be synergistic with chemotherapy, and has been used before, during, and after chemotherapy.

“Chemoradiotherapy” or “radiochemotherapeutic treatment” refers to the combination of chemotherapy and radiotherapy to treat cancer. In the art it can be also referred to radiochemotherapy and chemoradiation. In this type of therapy, chemotherapy and radiotherapy can be concurrent (i.e. applied together) or sequential (applied one after the other). In a referred embodiment of the invention, concurrent chemoradiotherapy is applied. The chemotherapy component can be or include a radiosensitizing agent. Chemoradiotherapy can be used as a neoadjuvant therapy before surgery. Concurrent Chemoradiotherapy is particularly suited to the treatment of head and neck cancers.

### METHODS OF THE INVENTION

The invention stems from the discovery by the inventors that the labelling pattern of CENP-A is a valuable biomarker of neoplastic state and also a predictive biomarker of cancer curability.

More specifically, the nuclear pattern of CENP-A labelling has been found associated for the first time to oncogenic transformation and cancer malignancy. Specific features are also found predictive of responsiveness of cancer to treatments.

### Method of Identifying an Oncogenic Transformation

Inventors have been able to identify and to select specific nuclear patterns of CENP-A labelling that are associated with the presence of a malignant lesion in a wide variety of tissue sample.

Accordingly, in a first aspect, the present invention relates to an in vitro method for identifying an oncogenic transformation in a tissue sample from a subject suspected to be afflicted with a cancer, comprising a step of determining a nuclear pattern of CENP-A labelling in cells from said tissue sample.

In an embodiment, the present invention relates to an in vitro method for identifying an oncogenic transformation in a tissue sample from a subject suspected to be afflicted with a cancer, comprising:
a step of labelling said tissue sample for CENP-A protein or for an homolog thereof,
a step of determining a nuclear pattern of CENP-A labelling in cells from said tissue sample.

More particularly, inventors have observed that in normal tissues or in benign lesions, CENP-A is found to cluster into foci, inside but specifically at the periphery of the nucleus, and optionally at the periphery of the nucleolus. Said clustering has been found to correspond to the grouping of regions from different chromosomes where CENP-A is enriched, as shown by the inventors. Conversely, this specific localization is found altered in tissue sample originating from cancer tissue samples, in which an at least partial delocalization of foci inside the nucleus is noticed. Also, in an embodiment, in said in vitro method for identifying an oncogenic transformation in a tissue sample from a subject, determining a nuclear pattern of CENP-A labelling comprises determining a presence and a subnuclear distribution of CENP-A foci.

As stated above and shown in experimental section, detecting intranuclear CENP-A foci distributed only at the nuclear periphery, and optionally nucleolar periphery, is found specific of tissues with no oncogenic transformation. Also, in an even more particular embodiment of the method for determining an oncogenic transformation in a tissue sample of a subject according to the invention, detecting intranuclear CENP-A foci which are distributed only at the nuclear periphery, and optionally nucleolar periphery, is indicative that no oncogenic transformation occurred in said sample and that, therefore, tissue sample does not display (or originate from) or a cancerous lesion thereby indicating that the subject is likely not suffering from a cancer in relation with the tissue sample subjected to the CENP-A labelling.

Inventors have studied CENP-A nuclear pattern of a large number of tissue samples from non-tumoral and tumoral tissues. They have shown that in tumoral tissue sample, at least a partial delocalization of CENP-A foci is observed which can be accompanied with an increase of the mean number of foci per nucleus and/or an alteration in their size and shape. Without wishing to be bound by any theory, these changes could be linked with a decrease and/or an alteration of CENP-A clustering which has been evidenced by the inventors. More particularly, in tumoral tissues, the CENP-A foci are found to be smaller and display a less regular round shape than observed in non-tumoral tissues. Furthermore, in some instances, an overall alteration of homogeneity of CENP-A labelling is observed both at the level of nucleus and at the level of the tissue (e.g. a different pattern is observed within the same sample from a cell to another) in terms of mean number per nucleus, size, and/or shape of CNEP-A foci. Accordingly, in an even more particular embodiment of the in vitro method according to the invention, determining a nuclear pattern of CENP-A labelling comprises determining, in said tissue sample:
the mean number of CENP-A foci per nucleus,
the size and/or shape of CENP-A foci, and/or
the intra-cell or intra-tissue heterogeneity of CENP-A labelling.

Based on their extensive analysis of tissue samples, Inventors have found that non tumoral tissues are characterized as displaying a mean number of CENP-A foci of between 9 to 18 foci per whole nucleus. Noticeably, this mean number is lower than the 46 foci that would be expected assuming that all centromeres were separated and labelled. Without being bound to any theory, it can therefore be estimated that foci that are detected are formed through the clustering of centromeric regions of 2 to 5 chromosomes within normal, not transformed cells.

Such a mean number of 9 to 18 foci per whole nucleus was extrapolated from the detection in IHC staining of between 4 to 8 foci per 3 micrometers thick section and was further confirmed by analyzing immunofluorescence staining with confocal microscopy.

Hence, in an embodiment of the method of the invention, detecting a mean from 9 to 18 intranuclear CENP-A foci per whole nucleus is indicative that no oncogenic transformation occurred in said sample and that, therefore, the tissue sample does not display a cancerous lesion, thereby indicating that the subject is likely not suffering from a cancer in relation with the tissue sample subjected to CENP-A labelling. In a more particular embodiment of the method according to the invention, a mean of 9, 10 11, 12, 13, 14, 15, 16, 17, or of 18 CENP-A foci per whole nucleus is indicative that no oncogenic transformation occurred in said sample and that, therefore, tissue sample does not display a cancerous lesion, thereby indicating that the subject is likely not suffering from a cancer in relation with the tissue sample subjected to CENP-A labelling. In another particular embodiment of the method according to the invention, detecting a mean from 4 to 8 intranuclear CENP-A foci per 3 micrometers thick section in IHC staining is indicative that no oncogenic transformation occurred in said sample and that, therefore, tissue sample does not display a cancerous lesion, thereby indicating that the subject is likely not suffering from a cancer in relation with the tissue sample subjected to CENP-A IHC staining. In an even more particular embodiment, detecting a mean of 10 intranuclear CENP-A foci per whole nucleus in a breast tissue sample is indicative that no oncogenic transformation occurred in said sample and that, therefore, tissue sample does not display a cancerous lesion, thereby indicating that the subject is likely not suffering from a breast cancer. The number of foci per whole nucleus can easily be determined from IHC staining by the following formula:

$$number of CENP - A foci per nucleus = detected number of foci \times \frac{cell nucleus diameter}{IHC section thickness}$$

and assuming that cell nucleus has a diameter of about 7 μm:

$$number of CENP - A foci per nucleus = detected number of foci \times \frac{7 μm}{IHC section thickness}$$

In a very particular embodiment of the invention, a tissue sample which does not display a cancerous lesion displays CENP-A foci which are distributed only at the nuclear periphery, and optionally nucleolar periphery, and found in a mean number of any one of the embodiments detailed in the preceding paragraph. In an even more particular embodiment of the in vitro method for identifying an oncogenic transformation in a tissue sample from a subject comprises determining a presence and a subnuclear distribution of CENP-A foci only at the nuclear periphery, and optionally nucleolar periphery, and in a mean number of from 9 to 18 intranuclear CENP-A foci per whole nucleus, such features being indicative that, consequently, no oncogenic transformation occurred in said sample and that, therefore, tissue sample does not display a cancerous lesion, thereby indicating that the subject is likely not suffering from a cancer in relation with the tissue sample subjected to CENP-A labelling.

Inventors have observed that CENP-A pattern of all tested tumoral tissue sample differs from the above described non tumoral pattern. In said tissues, the CENP-A foci do not localize strictly at the nuclear periphery anymore and rather localize inside the nucleus, even if few foci can still be observed at or in the vicinity of the nuclear periphery. In some instance, even no more CENP-A foci are detected in the nucleus, but a diffuse labelling of the nucleus is detected. Also, in another embodiment of the method according to the invention, when determining a nuclear pattern of CENP-A in a tissue sample of a subject comprises:
detecting no intranuclear CENP-A foci, or
detecting intranuclear CENP-A foci, at least a part thereof being not distributed at the nuclear periphery or at the nucleolar periphery,
then, said nuclear pattern of CENP-A is indicative that an oncogenic transformation occurred in said tissue sample, which therefore is at risk to display a malignant lesion, thereby indicating that the subject is at risk of suffering from a cancer in relation with the tissue sample subjected to CENP-A labelling.

In another embodiment of the method according to the invention when a mean number of less than 9 or of more than 18 CENP-A foci in nuclei of cells is detected in the labelled tissue sample, then said nuclear pattern of CENP-A is indicative that an oncogenic transformation occurred in said tissue sample, which therefore is at risk to display a malignant lesion, thereby indicating that the subject is suffering from a cancer in relation with the tissue sample subjected to CENP-A labelling. As mentioned above said number of foci in the nucleus can be extrapolated from the mean number that is observed in an IHC section of a tissue sample, or through 3D acquisition in confocal microscopy experiments.

In another embodiment of the method according to the invention when at least a part of CENP-A foci in the nucleus are less than 0.6 μm, less than 0.5 μm even more preferably less than 0.4 μm, in at least one of their dimensions in the labelled tissue sample, then said nuclear pattern of CENP-A is indicative that an oncogenic transformation occurred in said tissue sample, which therefore is at risk to display a malignant lesion, thereby indicating that the subject is suffering from a cancer in relation with the tissue sample subjected to CENP-A labelling.

Interestingly, inventors have observed that alterations in CENP-A nuclear pattern is more prominent in the most malignant lesions, or lesion of the most advanced stages. In a particular embodiment, a decreased clustering (an increase of foci number and a decrease of their size in relation with the normal tissues) allows to discriminate between benign and neoplastic lesions: the less a tissue exhibits CENP-A clustering and nuclear periphery localization, the more the lesion is found invasive. Also, loss of CENP-A localization at nuclear periphery associated with an heterogeneity in terms of number, size and shape across the nuclei of tissue sample and within a nucleus allow to discriminate, between neoplastic lesions, the non-invasive lesions from the invasive ones, that is the more malignant one form the others, an homogenous CENP-A pattern labelling being found associated by the inventors with the non- or less-invasive lesion.

In a particular embodiment, in regard with the number of the CENP-A, the number of the foci is defined as homogenous when at least 80% of the nucleus within the tested tissue sample present a number of between 9 to 18 foci of CENP-A.

In another particular embodiment, in regard with the shape of the CENP-A foci, it is defined as homogenous when at least 80% of the foci within each nucleus within the tested tissue sample present the same shape.

In another particular embodiment, at the level of the tissue sample, it is defined as homogenous when at least 80% of the of cells display a same CENP-A pattern in regard with, foci number, foci shape, and foci localization.

In a further particular embodiment, in regard the nuclear sublocalization of CENP-A foci, it is defined as homogenous when at least 80% of the nucleus within the tested tissue sample present the same proportion of foci at the periphery or within the nucleus.

In another particular embodiment homogeneity (or heterogeneity) of a sample labelled for CENP-A can be determined by any of the quantitative methods as described in Potts et al. (2012), such as well-known methods from the art using the Shannon's or Simpson's indices, or even the methods describes in Potts et al. (2012).

Method of Prognosing Response to Treatments of a Subject with a Cancer

Inventors have been further able to identify a specific CENP-A nuclear pattern that allows to discriminate cancer lesions that are responsive to cancer treatments from cancer lesions that are not responsive to such treatments. Indeed, retrospective analysis of tissue samples, show that amongst treated subjects, those for whose tissue samples present said specific CENP-A nuclear pattern had a better overall survival at 5 years.

Tissue samples wherein
at least a part of CENP-A intranuclear foci are not distributed at the nuclear periphery or at the nucleolar periphery, and
an homogeneous intra-cell CENP-A labelling in term of size, number, and/or labelling intensity of CENP-A foci, and
an homogeneous intra-tissue CENP-A labelling in term of size, number, and/or labelling intensity of CENP-A foci,
have been found to correspond to samples from malignant lesions which are responsive to radiotherapy, chemotherapy and/or concurrent chemoradiation therapy.

Conversely, tissue samples wherein
at least a part of CENP-A intranuclear foci are not distributed at the nuclear periphery or at the nucleolar periphery, and
an intra-cell or an inter-cell heterogeneity in the size, shape, or number of said foci is detected,
correspond to samples from malignant lesions which are at risk of being not responsive to radiotherapy, chemotherapy and/or concurrent chemoradiation therapy.

Also, in a second aspect, the present invention relates to an in vitro method for identifying an oncogenic transformation in a tissue sample from a subject suspected to be afflicted with a cancer, comprising a step of determining a nuclear pattern of CENP-A labelling in cells from said tissue sample, wherein determining a nuclear pattern of CENP-A comprises:
detecting intranuclear CENP-A foci, at least a part thereof being not distributed at the nuclear periphery or at the nucleolar periphery, and
detecting an homogeneous intra-cell CENP-A labelling in term of size, number, and/or labelling intensity of CENP-A foci, and
detecting an homogeneous intra-tissue CENP-A labelling in term of size, number, and/or labelling intensity of CENP-A foci,
which is indicative that sample tissue displays a malignant lesion which is responsive to radiotherapy, chemotherapy and/or concurrent chemoradiation therapy.

Alternatively, in this second aspect, the present invention relates to an in vitro method for identifying an oncogenic transformation in a tissue sample from a subject suspected to be afflicted with a cancer, comprising a step of determining a nuclear pattern of CENP-A labelling in cells from said tissue sample, wherein determining a nuclear pattern of CENP-A comprises:
detecting intranuclear CENP-A foci, at least a part thereof being not distributed at the nuclear periphery or at the nucleolar periphery, and
an intra-cell or an inter-cell heterogeneity in the size, shape, or number of said foci is detected,
which is indicative that sample that tissue sample displays a malignant lesion which is at risk of being not responsive to radiotherapy, chemotherapy and/or concurrent chemoradiation therapy.

In a particular embodiment of this method, subject is suspected to be afflicted from head and neck cancer, breast cancer such as triple negative breast cancer, ductal carcinoma in situ and invasive ductal carcinoma, prostate cancer, colorectal cancer, pancreas cancer, ovary cancer, glioblastoma, lung cancer, non-small cell lung cancer, ovarian cancer, bladder cancer, and cervical cancer, and detecting a nuclear pattern of CENP-A labelling in a tissue sample from the subject which comprises
detecting intranuclear CENP-A foci, at least a part thereof being not distributed at the nuclear periphery or at the nucleolar periphery, and
detecting an homogeneous intra-cell CENP-A labelling in term of size, number, and/or labelling intensity of CENP-A foci, and
detecting an homogeneous intra-tissue CENP-A labelling in term of size, number, and/or labelling intensity of CENP-A foci,
is indicative that sample tissue displays a malignant lesion corresponding to said cancer which is responsive to chemotherapy, radiotherapy and/or concurrent chemoradiation therapy.

In an even more particular embodiment of this method, the subject is suspected to be afflicted from head and neck cancer, and detecting a nuclear pattern of CENP-A labelling in a tissue sample from the subject which comprises
detecting intranuclear CENP-A foci, at least a part thereof being not distributed at the nuclear periphery or at the nucleolar periphery, and
detecting an homogeneous intra-cell CENP-A labelling in term of size, number, and/or labelling intensity of CENP-A foci, and
detecting an homogeneous intra-tissue CENP-A labelling in term of size, number, and/or labelling intensity of CENP-A foci,
is indicative that sample tissue displays a malignant lesion corresponding to said cancer which is responsive to chemotherapy, radiotherapy and/or concurrent chemoradiation therapy.

As exemplified in the experimental section, said CENP-A nuclear pattern is a biomarker which displays a sensitivity of 63.2%, and a specificity of 96%.

Also, in an embodiment, the invention relates to a method of prognosing the curability of a cancer by radiotherapy, chemotherapy and/or concurrent chemoradiation therapy said method comprising determining a nuclear pattern of CENP-A in a tissue sample from said cancer which comprises:
detecting intranuclear CENP-A foci, at least a part thereof being not distributed at the nuclear periphery or at the nucleolar periphery, and
detecting an homogeneous intra-cell CENP-A labelling in term of size, number, and/or labelling intensity of CENP-A foci, and
detecting an homogeneous intra-tissue CENP-A labelling in term of size, number, and/or labelling intensity of CENP-A foci,
and said nuclear pattern is indicative that cancer is responsive to radiotherapy, chemotherapy and/or concurrent chemoradiation therapy. More particularly, in said embodiment, cancer is selected from head and neck cancer, breast cancer such as triple negative breast cancer, ductal carcinoma in situ and invasive ductal carcinoma, prostate cancer, colorectal cancer, pancreas cancer, ovary cancer, glioblastoma, lung cancer, non-small cell lung cancer, ovarian cancer, bladder cancer, and cervical cancer.

Also, in a very particular embodiment, the invention relates to a method of prognosing the curability of a head and neck cancer by radiotherapy, chemotherapy and/or concurrent chemoradiation therapy said method comprising determining a nuclear pattern of CENP-A in a tissue sample from said cancer which comprises:
detecting intranuclear CENP-A foci, at least a part thereof being not distributed at the nuclear periphery or at the nucleolar periphery, and
detecting an homogeneous intra-cell CENP-A labelling in term of size, number, and/or labelling intensity of CENP-A foci, and
detecting an homogeneous intra-tissue CENP-A labelling in term of size, number, and/or labelling intensity of CENP-A foci,
and said nuclear pattern indicative that said head and neck cancer is responsive to radiotherapy, chemotherapy and/or concurrent chemoradiation therapy.

In a particular embodiment the invention relates to a method of prognosing the curability by radiotherapy of a cancer selected from head and neck cancer, breast cancer such as triple negative breast cancer, ductal carcinoma in situ and invasive ductal carcinoma, prostate cancer, colorectal cancer, pancreas cancer, ovary cancer, glioblastoma, lung cancer, non-small cell lung cancer, ovarian cancer, bladder cancer, and cervical cancer, said method comprising determining a nuclear pattern of CENP-A in a tissue sample from said cancer which comprises:
detecting intranuclear CENP-A foci, at least a part thereof being not distributed at the nuclear periphery or at the nucleolar periphery, and
detecting an homogeneous intra-cell CENP-A labelling in term of size, number, and/or labelling intensity of CENP-A foci, and
detecting an homogeneous intra-tissue CENP-A labelling in term of size, number, and/or labelling intensity of CENP-A foci,
and is indicative that said cancer is responsive to radiotherapy.

In a particular embodiment the invention relates to a method of prognosing the curability by concurrent chemoradiation therapy of a cancer selected from head and neck cancer, breast cancer such as triple negative breast cancer, ductal carcinoma in situ and invasive ductal carcinoma, prostate cancer, colorectal cancer, pancreas cancer, ovary cancer, glioblastoma, lung cancer, non-small cell lung cancer, ovarian cancer, bladder cancer, and cervical cancer, said method comprising determining a nuclear pattern of CENP-A in a tissue sample from said cancer which comprises:
detecting intranuclear CENP-A foci, at least a part thereof being not distributed at the nuclear periphery or at the nucleolar periphery, and
detecting an homogeneous intra-cell CENP-A labelling in term of size, number, and/or labelling intensity of CENP-A foci, and
detecting an homogeneous intra-tissue CENP-A labelling in term of size, number, and/or labelling intensity of CENP-A foci,
and said nuclear pattern is indicative that said cancer is responsive to concurrent chemoradiation therapy.

In a particular embodiment the invention relates to a method of prognosing the curability by concurrent chemotherapy of a cancer selected from head and neck cancer, breast cancer such as triple negative breast cancer, ductal carcinoma in situ and invasive ductal carcinoma, prostate cancer, colorectal cancer, pancreas cancer, ovary cancer, glioblastoma, lung cancer, non-small cell lung cancer, ovarian cancer, bladder cancer, and cervical cancer, said method comprising determining a nuclear pattern of CENP-A in a tissue sample from said cancer which comprises:
detecting intranuclear CENP-A foci, at least a part thereof being not distributed at the nuclear periphery or at the nucleolar periphery, and
detecting an homogeneous intra-cell CENP-A labelling in term of size, number, and/or labelling intensity of CENP-A foci, and
detecting an homogeneous intra-tissue CENP-A labelling in term of size, number, and/or labelling intensity of CENP-A foci,
and said nuclear is indicative that said cancer is responsive to concurrent chemotherapy.

Also, in an embodiment, the invention relates to a method of prognosing the curability of a cancer by radiotherapy, chemotherapy and/or concurrent chemoradiation therapy said method comprising determining a nuclear pattern of CENP-A in a tissue sample from said cancer which comprises:
detecting intranuclear CENP-A foci, at least a part thereof being not distributed at the nuclear periphery or at the nucleolar periphery, and
an intra-cell or an inter-cell heterogeneity in the size, shape, or number of said foci is detected,
which said nuclear pattern is indicative that sample tissue displays a malignant lesion which is at risk of being not responsive to radiotherapy, chemotherapy and/or concurrent chemoradiation therapy. More particularly, in said embodiment, cancer is selected from head and neck cancer, breast cancer such as triple negative breast cancer, ductal carcinoma in situ and invasive ductal carcinoma, prostate cancer, colorectal cancer, pancreas cancer, ovary cancer, glioblastoma, lung cancer, non-small cell lung cancer, ovarian cancer, bladder cancer, and cervical cancer.

Also, in a very particular embodiment, the invention relates to a method of prognosing the curability of a head and neck cancer by radiotherapy, chemotherapy and/or concurrent chemoradiation therapy said method comprising determining a nuclear pattern of CENP-A in a tissue sample from said cancer which comprises:
detecting intranuclear CENP-A foci, at least a part thereof being not distributed at the nuclear periphery or at the nucleolar periphery, and
an intra-cell or an inter-cell heterogeneity in the size, shape, or number of said foci is detected,
and said nuclear pattern is indicative that said head and neck cancer is at risk of being not responsive to radiotherapy, chemotherapy and/or concurrent chemoradiation therapy.

In a particular embodiment the invention relates to a method of prognosing the curability by radiotherapy of a cancer selected from head and neck cancer, breast cancer such as triple negative breast cancer, ductal carcinoma in situ and invasive ductal carcinoma, prostate cancer, colorectal cancer, pancreas cancer, ovary cancer, glioblastoma, lung cancer, non-small cell lung cancer, ovarian cancer, bladder cancer, and cervical cancer, said method comprising determining a nuclear pattern of CENP-A in a tissue sample from said cancer which comprises:
detecting intranuclear CENP-A foci, at least a part thereof being not distributed at the nuclear periphery or at the nucleolar periphery, and
an intra-cell or an inter-cell heterogeneity in the size, shape, or number of said foci is detected,
and said nuclear pattern is indicative that said cancer which is at risk of being not responsive to radiotherapy.

In a particular embodiment the invention relates to a method of prognosing the curability by concurrent chemoradiation therapy of a cancer selected from head and neck cancer, breast cancer such triple negative breast cancer, ductal carcinoma in situ and invasive ductal carcinoma, prostate cancer, colorectal cancer, pancreas cancer, ovary cancer, glioblastoma, lung cancer, non-small cell lung cancer, ovarian cancer, bladder cancer, and cervical cancer, said method comprising determining a nuclear pattern of CENP-A in a tissue sample from said cancer which comprises:
detecting intranuclear CENP-A foci, at least a part thereof being not distributed at the nuclear periphery or at the nucleolar periphery, and
an intra-cell or an inter-cell heterogeneity in the size, shape, or number of said foci is detected,
and said nuclear pattern is indicative that said cancer which is at risk of being not responsive to concurrent chemoradiation therapy.

In a particular embodiment the invention relates to a method of prognosing the curability by chemotherapy of a cancer selected from head and neck cancer, breast cancer such as triple negative breast cancer, ductal carcinoma in situ and invasive ductal carcinoma, prostate cancer, colorectal cancer, pancreas cancer, ovary cancer, glioblastoma, lung cancer, non-small cell lung cancer, ovarian cancer, bladder cancer, and cervical cancer, said method comprising determining a nuclear pattern of CENP-A in a tissue sample from said cancer which comprises:
detecting intranuclear CENP-A foci, at least a part thereof being not distributed at the nuclear periphery or at the nucleolar periphery, and
an intra-cell or an inter-cell heterogeneity in the size, shape, or number of said foci is detected,
and said nuclear pattern is indicative that said cancer is not responsive to chemotherapy.

In a very particular embodiment, the invention relates to a method of prognosing the overall survival of a subject treated or to be treated by radiotherapy, chemotherapy and/or concurrent chemoradiation therapy and suffering from a cancer said method comprising determining a nuclear pattern of CENP-A in a tissue sample from said cancer which comprises:
detecting intranuclear CENP-A foci, at least a part thereof being not distributed at the nuclear periphery or at the nucleolar periphery, and
detecting an homogeneous intra-cell CENP-A labelling in term of size, number, and/or labelling intensity of CENP-A foci, and
detecting an homogeneous intra-tissue CENP-A labelling in term of size, number, and/or labelling intensity of CENP-A foci,
and said nuclear pattern is indicative of probability of at least 70% probability of a survival to 5 years.

In a more particular embodiment, the invention relates to a method of prognosing the overall survival of a subject treated or to be treated by radiotherapy, chemotherapy and/or concurrent chemoradiation therapy and suffering from a cancer selected from head and neck cancer, breast cancer such as triple negative breast cancer, ductal carcinoma in situ and invasive ductal carcinoma, prostate cancer, colorectal cancer, pancreas cancer, ovary cancer, glioblastoma, lung cancer, non-small cell lung cancer, ovarian cancer, bladder cancer, and cervical cancer, said method comprising determining a nuclear pattern of CENP-A in a tissue sample from said cancer which comprises:
detecting intranuclear CENP-A foci, at least a part thereof being not distributed at the nuclear periphery or at the nucleolar periphery, and
detecting an homogeneous intra-cell CENP-A labelling in term of size, number, and/or labelling intensity of CENP-A foci, and
detecting an homogeneous intra-tissue CENP-A labelling in term of size, number, and/or labelling intensity of CENP-A foci,
and said nuclear pattern is indicative of probability of at least 70% of a survival to 5 years.

In a more particular embodiment, the invention relates to a method of prognosing the overall survival of a subject treated or to be treated by radiotherapy, chemotherapy and/or concurrent chemoradiation therapy and suffering from head and neck cancer said method comprising determining a nuclear pattern of CENP-A in a tissue sample from said cancer which comprises:
detecting intranuclear CENP-A foci, at least a part thereof being not distributed at the nuclear periphery or at the nucleolar periphery, and
detecting an homogeneous intra-cell CENP-A labelling in term of size, number, and/or labelling intensity of CENP-A foci, and
detecting an homogeneous intra-tissue CENP-A labelling in term of size, number, and/or labelling intensity of CENP-A foci,
and said nuclear pattern is indicative of probability of at least 70% of a survival to 5 years.
   Method of Prognosing Response to Treatments and Adapting Cancer Treatment Strategy of a Subject with a Cancer

Determining the responsiveness of a malignant lesion is of an outmost advantage in the field of cancer management. Indeed, it can help the clinicians to select appropriate therapy for the patients in such a manner that they will potentially benefit from prevention of recurrence of tumor or of control of said tumor or malignant lesion. Avoiding use of inappropriate or ineffective therapy can save time and life duration expectancy for the patient, avoid or delay invasive and potentially debilitating surgical intervention, or avoid unnecessarily worsening of quality of life of a subject. From a societal point of view, prognostic tools of responsiveness to treatment are useful in avoiding wasting resources with costly and inappropriate treatments in hopeless situations or undertreatment of the disease. CENP-A nuclear patterns as identified by the inventors being linked to responsiveness of the cancer to chemotherapy, radiotherapy and/or concurrent chemoradiation, therefore provide a valuable biomarker for disease management.

Also, in a third aspect, invention relates to method of treating a subject suffering from a cancer wherein treatment options are chosen depending on the CENP-A pattern that is observed in a tissue sample from cancer of the subject.

In a particular embodiment, said method of treating comprises determining a nuclear pattern of CENP-A in a tissue sample from said cancer which comprises:
detecting intranuclear CENP-A foci, at least a part thereof being not distributed at the nuclear periphery or at the nucleolar periphery, and
detecting an homogeneous intra-cell CENP-A labelling in term of size, number, and/or labelling intensity of CENP-A foci, and
detecting an homogeneous intra-tissue CENP-A labelling in term of size, number, and/or labelling intensity of CENP-A foci,
said subject being then diagnosticated as suffering from a cancer responsive to chemotherapy and/or radiotherapy and/or concurrent chemoradiation therapy, said method of treating cancer comprising prioritizing a chemotherapeutic, a radiotherapeutic or a concurrent chemoradiation therapy over surgical resection of tumorous lesion in the subject.

In a more particular embodiment of said method of treating, prioritizing a chemotherapeutic, a radiotherapeutic or a concurrent chemoradiation therapy over surgical resection of tumorous lesion in the subject comprises delaying said surgical resection till the cancer is diagnosed as having evolved toward resistant to said treatment. In particular embodiment, of said method of treating, prioritizing a chemotherapeutic, a radiotherapeutic or a concurrent chemoradiation therapy over surgical resection of tumorous lesion in the subject consists in applying a chemotherapeutic, a radiotherapeutic or a concurrent chemoradiation therapy before performing surgical resection of tumorous lesion. In an even more particular embodiment, said cancer is selected from head and neck cancer, breast cancer such triple negative breast cancer, ductal carcinoma in situ and invasive ductal carcinoma, prostate cancer, colorectal cancer, pancreas cancer, ovary cancer, glioblastoma, lung cancer, non-small cell lung cancer, ovarian cancer, bladder cancer, and cervical cancer. In a further particular embodiment, of said method of treating, cancer is a head and neck cancer and a radiotherapeutic or a concurrent chemoradiation therapy is prioritized over surgical resection of tumorous lesion in the subject.

In another particular embodiment, said method of treating comprises determining a nuclear pattern of CENP-A in a tissue sample from said cancer, which comprises
at least a part of CENP-A intranuclear foci are not distributed at the nuclear periphery or at the nucleolar periphery, and
an intra-cell or an inter-cell heterogeneity in the size, shape, or number of said foci is detected,
the subject being then diagnosticated as suffering from a cancer likely resistant to chemotherapy and/or radiotherapy and/or concurrent chemoradiation therapy
and said method of treating comprises prioritizing surgical resection of tumorous lesion in the subject and/or not subjecting said subject to a chemotherapeutic, a radiotherapeutic or a concurrent chemoradiation therapy.

### Method of Preparing a Tissue Sample

Also provided is a method of preparing a tissue sample useful analyzing the features of a CENP-A labelling in a tissue sample. Indeed, inventors have been able to identify conditions that allow an accurate detection and analysis of the specific CENP-A nuclear pattern thereby allowing to implement diagnostic and prognostic methods according to the invention.

Accordingly, in a particular embodiment, invention also relates to methods for preparing a tissue sample useful for analyzing the features of the CENP-A pattern in cells from said tissue sample, said method comprising:
a) producing section of the tissue sample to be analyzed,
b) preparing a low formalin fixative solution,
c) incubating the sample with the fixative solution,
wherein the tissue sample resulting from c) comprise centromeric structures that are detectable through CENP-A specific antibodies,
d) analyzing CENP-A pattern in cells from said tissue sample.

In an even more particular embodiment of said method of preparing a tissue sample, the low formalin said low formalin fixative solution contains less than 3.5% formalin, less than 3% preferably less than 2%, even more preferably less 1% or less formalin or even contains no formalin. A particularly preferred fixative solution is Alcohol-Formalin-Acetic acid (AFA) which contains 1% formalin. In a more particular embodiment, the fixative solution is AFA containing less than 1% formalin and the labelling and detection method of CENP-A nuclear pattern is an immunohistochemistry chromogenic labelling of the tissue sample.

In another particular embodiment, the fixative solution is a paraformaldehyde solution, and the labelling and detection method of CENP-A nuclear pattern is immunofluorescence.

### Automation of Methods of the Invention

The determination of features of the CENP-A pattern as identified by the inventors (number, shape, subnuclear localization, homogeneity) can easily be implemented by a computer. For example, a computer can be used to specifically analyze tissue samples labelled for CENP-A and generate a report of the localization of the CENP-A foci, the number of CENP-A foci per whole nucleus, the shape of the CENP-A foci, the intensity of the labelling, and/or the homogeneity of the CENP-A labelling, optionally with the corresponding images.

Moreover, the determination of features of the CENP-A pattern as identified by the inventors (number, shape, subnuclear localization, homogeneity) can be implemented in an automated analysis of tissue samples labelled for CENP-A. Using artificial intelligence and/or machine learning, as e.g. deep learning methods, tools can be trained to predict the neoplastic nature as well as the sensitivity or resistance to therapy based on CENP-A staining. This is of particular interest in the field of cancer diagnosis and prognosis and results in the increase of the consistency between different readers and laboratories and boosts the speed and efficiency of the diagnostic procedure which can be lengthy otherwise.

Also, in a particular embodiment, the methods of the invention are implemented by a computer device which comprises processors suitable to:
i) analyze images from tissue samples labelled for CENP-A and determine
   localization of the CENP-A foci,
   number of CENP-A foci per whole nucleus,
   shape of the CENP-A foci,
   intensity of the labelling, and/or
   homogeneity of the CENP-A labelling;
ii) assign the tissue sample to one of the CENP-A patterns as described herein; and
iii) display whether the tissue sample displays a cancerous lesion, and/or whether, when the tissue sample displays such a lesion, it is responsive or likely not responsive to chemotherapy, radiotherapy and/or concurrent chemoradiotherapy.

### Kits of the Invention

In a fourth aspect, the invention also relates to a kit suitable to implement any of the methods of the invention.

In an embodiment said kit comprises:
At least one antibody, or a fragment thereof, preferably a monoclonal antibody, or a fragment thereof, a recombinant antibody or a fragment thereof, a nanobody or a fragment thereof, or an aptamer, which binds CENP-A,
Instruction for labelling a tissue sample with said antibody, or a fragment thereof, and/or
Instruction for analyzing the nuclear pattern of CENP-A labelling to implement the method of any one of the methods of the inventions, in any of their embodiments.

Said kit can therefore further comprise instruction allowing classification of the subject as afflicted with malignant carcinoma, and/or chemotherapeutic, a radiotherapeutic or a concurrent chemoradiation therapy resistant carcinoma.

In a particular embodiment said kit further comprises a fixative solution which contains less than 3.5% formalin, less than 3% preferably less than 2%, even more preferably less than 1% formalin or even which contains no formalin.

In a very particular embodiment said kit further comprises at least one control IHC sample corresponding to a normal sample, displaying features of non-cancerous tissue as exposed above, or to the different CENP-A patterns associated or not with sensitivity to treatment. In a more particular embodiment, the fixative solution is AFA containing less than 1% formalin and the labelling and detection method of CENP-A nuclear pattern is an immunohistochemistry chromogenic labelling of the tissue sample.

In another particular embodiment, the fixative solution is a paraformaldehyde solution, for example a 2% paraformaldehyde solution and the labelling and detection method of CENP-A nuclear pattern is immunofluorescence.

### CENP-A Protein Labelling Means

Any means that allows the specific detection and labelling of CENP-A protein or of a homolog thereof in a tissue sample is suitable for implementing methods of the invention and to be used in the kits according to the invention. Preferably, said means should allow to distinguish CENP-A clusterization in normal, non-cancerous, tissues or cells as evidenced and described herein. This can be easily tested by performing, e.g., IHC chromogenic staining or confocal microscopy fluorescence experiments. In a preferred embodiment, CENP-A pattern is detected by means of an antibody, a fragment thereof or an aptamer.

As stated, said antibody or aptamer, is specific to CENP-A proteins and should therefore not detect proteins which might interfere with CENP-A patterns as disclosed herein. This could be tested by methods well known in the art, as, e.g., western blotting, ELISA.

### Antibody

In a preferred embodiment, said antibody specifically binds the CENP-A protein of SEQ ID NO: 1 or of SEQ ID NO: 2, or a homolog thereof. In another preferred embodiment, said antibody specifically binds the peptide of SEQ ID NO: 3 “PRRRSRKPEAPRRRSPS”, from amino acid residues 3 to 19 in CENP-A isoforms.

The terms “antibody”, “antibodies” or “immunoglobulin” are used interchangeably throughout this application. They should be construed in the broadest sense: these terms, as used herein, thus include monoclonal antibodies (e.g., full length or intact monoclonal antibodies), polyclonal antibodies, multivalent antibodies or multispecific antibodies (e.g., bispecific antibodies so long as they exhibit the desired biological activity) and functional fragments thereof.

The term “antibody” as used herein designates a polypeptide that exhibits binding specificity to a specific antigen. More particularly, an antibody (or “immunoglobulin”) consists of a glycoprotein comprising at least two heavy (H) chains and two light (L) chains interconnected by disulfide bonds.

Each heavy chain comprises a heavy chain variable region (or domain) (abbreviated herein as VH) and a heavy chain constant region (hereafter CH). Heavy chains are classified as gamma, mu, alpha, delta, or epsilon, and define the antibody's isotype as IgG, IgM, IgA, IgD, and IgE, respectively. The CH region of the immunoglobulin IgG, IgD, and IgA (γ, δ and α chains respectively) comprises three domains (CH1, CH2, and CH3) and a hinge region for added flexibility, while the CH region of the immunoglobulin IgM and IgE contains 4 domains (CH1, CH2, CH3, and CH4).

IgG antibodies are classified in four distinct subtypes, named IgG1, IgG2, lgG3 and lgG4. The structure of the hinge regions in the γ chain gives each of these subtypes its unique biological profile (even though there is about 95% similarity between their Fc regions, the structure of the hinge regions is relatively different).

Each light chain comprises a light chain variable region (abbreviated herein as VL) and a light chain constant region comprising only one domain (abbreviated as CL). There are two types of light chain in mammals: the kappa (κ) chain, encoded by the immunoglobulin kappa locus on chromosome 2, and the lambda (λ) chain, encoded by the immunoglobulin lambda locus on chromosome 22.

The VH and VL regions can be further subdivided into regions of hypervariability, termed “Complementarity Determining Regions” (CDR), which are primarily responsible for binding an antigen, and which are interspersed with regions that are more conserved, designated “Framework Regions” (FR). Each VH and VL is composed of three CDRs and four FRs, arranged from amino-terminus to carboxy-terminus in the following order: FR1, CDR1, FR2, CDR2, FR3, CDR3, FR4. The assignment of amino acid sequences to each domain is in accordance with well-known conventions (see e.g. Lefranc, M.-P., et al., (2003)). The functional ability of the antibody to bind a particular antigen depends on the variable regions of each light/heavy chain pair, and is largely determined by the CDRs. The variable region of the heavy chain differs in antibodies produced by different B cells, but is the same for all antibodies produced by a single B cell or B cell clone (or hybridoma). By contrast, the constant regions of the antibodies mediate the binding of the immunoglobulin to host tissues or factors, including various cells of the immune system (e.g. effector cells) and the first component (C1q) of the classical complement system.

“Antibody” as used herein also designates “single chain antibody” or sdAb, or related molecules, which refers to the single heavy chain variable domain (VHH) of antibodies of the type that can be found in camelids which are naturally devoid of light chains. Such single domain antibodies are also named nanobodies. They represent the smallest antibody fragments (around 14 kDa) able to preserve the binding affinity and specificity of the original whole antibody and they are appreciated for their structural stability and their simple engineering into reagents suitable for in vitro and in vivo applications (de Marco (2020)). They can be obtained by classical immunization of animals known to produce such antibodies, e.g. camelids or cartilaginous fishes with the desired antigen and subsequent isolation of the mRNA coding for heavy-chain antibodies. They can be also produced as “recombinant antibodies” and purified using genetic engineering as reviewed in de Marco (2020).

As used herein, the term “antibody fragment” intends to designate Fab, Fab′, F(ab′)2, scFv, dsFv, ds-scFv, dimers, minibodies, diabodies, multimers thereof or bispecific antibody fragments. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques. They are also referred to as “recombinant antibodies”. For example, scFvs, comprise the variable regions of the immunoglobulin heavy and light chain, covalently connected by a peptide linker. These “antibody fragments” are rather small in comparison with antibodies and generally retain specificity and affinity for antigen in a single polypeptide and can provide a convenient building block for larger, antigen-specific molecules.

“Antibody” as used herein also designates any combination of part(s) or of fragment(s) of antibodies as described above provided that it retains sufficient affinity constant and/or dissociation constant for CENP-A, as defined below. For example, both scFv fragments or VHH nanobodies can be linked to the Fc fragment of the desired species and keep their specificity and binding properties.

An “epitope” is the site on the antigen to which an antibody binds. It can be formed by contiguous residues or by non-contiguous residues brought into close proximity by the folding of an antigenic protein. In particular, an epitope can comprise a residue carrying a specific post-translational modification, e.g. a glycosylation or a phosphorylation, said specific post-translational modification ensuring specific reconnaissance by the antibody. For example, in the present case, the epitope which is recognized by the antibody is a group of contiguous residues or by non-contiguous amino acid residues of CENP-A brought into close proximity by the folding of an antigenic protein.

A “functional fragment” of an antibody means, in particular, an antibody fragment as defined above, with the same binding activity to CENP-A as the parental antibody.

In the context of the present invention, an antibody or fragment thereof is said to “recognize” or “bind” a peptide having a defined sequence if said antibody has an affinity constant Ka (which is the inverted dissociation constant, i.e., 1/Kd) higher than 10⁶ M⁻¹, preferably higher than 10⁷ M⁻¹, more preferably higher than 10⁸ M⁻¹ for said peptide. Also, in the context of the present invention, an antibody is said to “specifically bind” or to “specifically recognize” a peptide if said antibody or fragment thereof has an affinity constant Ka greater than 10⁷ M⁻¹, preferably greater than 10⁸ M⁻¹, more preferably greater than 10⁹ M⁻¹ for said peptide and even more preferably greater than 10¹⁰ M⁻¹ for said peptide and has an affinity constant Ka lower than 10⁵ M⁻¹ for all the other peptide.

This affinity can be measured for example by equilibrium dialysis or by fluorescence quenching, both technologies being routinely used in the art.

In a preferred embodiment, the antibody to be used in any of the methods of the invention binds CENP-A with a Kd of less than 10⁻⁷ M, preferably from less than 10⁻⁸ M. In a further preferred embodiment, the antibodies to be used in method of the invention bind CENP-A with a Kd of less than 10⁻⁹ M, preferably from less than 10⁻¹⁰ M.

Determining Kd or Ka of an antibody is well known from the skilled in the art.

The antibody of the methods of the invention may be monoclonal or polyclonal and may be of any species of origin, including (for example) mouse, rat, rabbit, horse, or human, or may be a chimeric antibody.

A “polyclonal antibody” as used herein, refers to an antibody that is obtained from different B cells. It typically includes various antibodies directed against various determinants, or epitopes, of the target antigen. Polyclonal antibodies that specifically bind CENP-A may be produced by standard antibody production methods, for example by i) immunizing a suitable animal (e.g., rabbit, goat, etc.) with CENP-A protein or homolog thereof (of e.g. of SEQ ID NO: 3) or with an immunogenic peptide (e.g. of SEQ ID NO: 3), ii) collecting immune serum from the animal, and iii) separating the polyclonal antibodies from the immune serum, in accordance with known procedures.

For example, the immunogenic peptide of SEQ ID NO: 3 may be used to produce the antibodies suitable to be used in method or kits of the invention. It will be appreciated by those of skill in the art that longer or shorter immunogenic peptides may also be employed.

An immunogenic peptide can be synthetized by conventional means and can be used to generate a polyclonal antibody suitable to be used in the method of the invention.

A “monoclonal antibody”, as used herein, means an antibody arising from a nearly homogeneous antibody population. The individual antibodies of a population are identical except for a few possible naturally-occurring mutations which can be found in minimal proportions. In other words, a monoclonal antibody consists of a homogeneous antibody arising from the growth of a single cell clone (for example a hybridoma, a eukaryotic host cell transfected with a DNA molecule coding for the homogeneous antibody, a prokaryotic host cell transfected with a DNA molecule coding for the homogeneous antibody, etc.) and is characterized by heavy chains of one and only one isotype and subtype, and light chains of only one type. Monoclonal antibodies are highly specific and are directed against a single epitope of an antigen. Monoclonal antibodies may be produced by a single clone of B cells or “hybridoma”. Monoclonal antibodies may also be recombinant, i.e., produced by protein engineering. The invention relates to monoclonal antibodies isolated or obtained by purification from natural sources or obtained by genetic recombination or chemical synthesis.

The monoclonal antibodies suitable for the method of the invention may be produced in a hybridoma cell line according to the well-known technique of Kohler and Milstein, 1975; Kohler and Milstein, 1976; see also, CURRENT PROTOCOLS IN MOLECULAR BIOLOGY, Ausubel et al. (1989). Monoclonal antibodies are preferably used in the method of classifying subject afflicted with solid cancer of the invention.

For example, a solution containing the appropriate antigen may be injected into a mouse or other species and, after a sufficient time (in keeping with conventional techniques), the animal is sacrificed, and spleen cells obtained. The spleen cells are then immortalized by fusing them with myeloma cells, typically in the presence of polyethylene glycol, to produce hybridoma cells. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal Fab fragments may also be produced in Escherichia coli by recombinant techniques known to those skilled in the art (W. Huse, 1989; Mullinax et al., 1990). If monoclonal antibodies of one isotype are preferred for a particular application, particular isotypes can be prepared directly, by selecting from the initial fusion, or prepared secondarily, from a parental hybridoma secreting a monoclonal antibody of different isotype (Steplewski, et al., 1985; Spira et al., 1984). Recombinant cells for producing an antibody suitable to be used in the method of the invention cells may be constructed by well-known techniques; for example, the antigen combining site of the monoclonal antibody can be cloned by PCR and single-chain antibodies produced as phage-displayed recombinant antibodies or soluble antibodies in E. coli.

Antibodies (or fragment thereof) to be used in the methods and kits of the invention specifically bind CENP-A. This specificity may be screened according to standard techniques (Czernik et al., 1991) such as ELISA. The antibodies may also be tested by western blotting. Antibodies may be further characterized via immunohistochemical (IHC) staining using normal tissues and identifying the CENP-A clusterization and pattern as exposed herein. IHC may be carried out on paraffin-embedded tissues according to well-known techniques, for example comprising the steps of: i) deparaffinizing tissue sections with xylene followed by ethanol; ii) hydrating in water then PBS; iii) unmasking antigen by heating slide in sodium citrate buffer; iv) incubating sections in hydrogen peroxide; v) blocking in blocking solution; vi) incubating slide in primary antibody and secondary antibody; and finally vii) detecting using ABC avidin/biotin method according to manufacturer's instructions (see ANTIBODIES: A LABORATORY MANUAL, Chapter 10, Harlow & Lane Eds., Cold Spring Harbor Laboratory (1988)). The antibodies may be further characterized by flow cytometry carried out according to standard methods (Chow et al., 2001).

Antibodies (or antibody fragment) specific to CENP-A can be detected to determine the CENP-A patterns as disclosed herein, in immunohistochemistry using numerous means well known form the skilled in the art. For example, they can be advantageously conjugated to fluorescent dyes (e.g. Alexa-488, Phycoerythrin (PE), Fluorescein isothiocyanate (FITC)). Said antibodies can also be biotinylated to be further revealed using horseradish coupled streptavidin or other equivalent means. Otherwise said antibodies can also be detected by an indirect method principle of which is well known from the skilled in the art; briefly in the indirect labelling IHC methods, antibodies specific CENP-A are used as primary antibody, is not conjugated and is allowed to react with cancer cell sample, and a labeled secondary antibody directed against isotype of the animal species in which the primary antibody has been raised which is conjugated either with a fluorescent dye or with an enzyme (such as, e.g., peroxidase, alkaline phosphatase or glucose oxidase) or a molecular prey (such a biotin) for enzymatic detection. The indirect method is known to be more sensitive because of signal amplification resulting from the binding of several secondary antibodies on the primary antibody. Chromogenic detection methods in either direct or indirect IHC rely on enzymes that convert soluble substrates into insoluble colored products. Antibodies can also be radiolabeled by methods well known in the art or barcoded (labeled with ssDNA or ds DNA oligonucleotides) e.g. as shown in Kohman and Church (2020).

In a preferred embodiment, the methods of the invention, in any of the above embodiments, is implemented by using antibody (ies) specific to CENP-A in immunohistochemical (IHC) staining of tissue sample, which is a currently used method in the field of diagnosis and reviewed, for example by Ramos-Vara and Miller (2014) and detailed in the experimental section. In a preferred embodiment, methods according to the inventions are implemented using a chromogenic IHC method.

In an even more preferred embodiment, in the methods of the invention, tissue sample are subjected to a fixation in low formalin fixative solution. Advantageously, such fixative solutions allow particularly clear and well-defined labelling of CENP-A. More preferably said low formalin fixative solution contains less than 3.5% formalin, less than 3% preferably less than 2%, even more preferably less than 1% formalin or even contains no formalin. A particularly preferred fixative solution is Alcohol-Formalin-Acetic acid (AFA) which contains 1% or less formalin. Accordingly, in a particular embodiment of any of the methods of the invention, the step of labelling said tissue sample for CENP-A protein or for an homolog thereof is performed on a tissue sample that have been previously fixed with a fixative containing less than 3.5% less than 3% preferably less than 2%, even more preferably less than 1% formalin or even containing no formalin, even more preferably in a chromogenic Immunohistochemical (IHC) method. in another particular embodiment of any of the methods of the invention, the step of labelling said tissue sample for CENP-A protein or for an homolog thereof is performed on a tissue sample that have been previously fixed with AFA, even more preferably in an chromogenic immunohistochemical (IHC) method.

Commercial CENP-A monoclonal and polyclonal antibodies can be used in the methods according to the invention (e.g., monoclonal antibody #ADI-KAM-CC006-E from Enzo life sciences or polyclonal antibody #2186 from Cell Signalling).

### Aptamer

In another embodiment, an aptamer is used to detect CENP-A in the methods and kits according to the invention (Neuberger et al., 1984). Said aptamer is preferably a nucleic acid-based aptamer (i.e. either RNA or DNA aptamer). Nucleic acid-based aptamers are being developed for a variety of diagnostic applications, including detection of a wide range of non-nucleic acid analytes (Conrad et al., 1996). Aptamers can be selected in vitro by the SELEX process from very large populations of random sequence oligomers (Ellington & Szostak, 1990). This well-established methodology selects aptamers based on their affinity for a specific target molecule. Aptamers can be selected against nearly any class of molecule including proteins, ranging from simple peptides to post-translationally modified proteins. The post-translational modifications potentially detectable by aptamers include a variety of common covalent modifications such as phosphorylation, glycosylation, and proteolytic cleavage and noncovalent modifications such as conformational changes due to binding of ligands (McCauley et al., 2003). Aptamers do not depend on immunological reaction of animals to be produced. Aptamers present the interest of being produced either by chemical synthesis or in bacteria and are thus more quickly and more reproducibly produced and available than antibodies. As antibodies, aptamers can be selected based upon their affinity for the ligand for which they are produced (see e.g. in Ahirwar et al., 2016). Example of production and uses of aptamers in so called aptohistochemistry in cancer are provided e.g., in Ahirwar et al. (2016) or Zamay et al. (2017). Aptamers can be conjugated, as antibodies do, to either fluorescent dyes or other detection means or streptavidin-based amplification techniques, and detected therefore in confocal fluorescence microscopy or chromogenic IHC.

### Examples

### Material and Methods

### Ethical Approval

All experiments on human tissues were performed retrospectively and in accordance with the French Bioethics Law 2004-800, the French National Institute of Cancer (INCa) Ethics Charter, and after approval by the Institut Curie review board.

Experimental procedures of xenograft experiments were specifically approved by the ethics committee of the Institut Curie CEEA-IC #118 (Authorization #21973-2019091218441437 v1-given by National Authority) in compliance with the international guidelines.

### Tissue Samples

Biopsies used in the study were fixed at collection in AFA (Alcohol, Formaldehyde and Acetic acid) with low formaldehyde content. 16 various types of primary malignant tumors (n=628) and 16 various types of normal tissues (n=249), collected from multi-center Departments of Pathology from 1978 to 2010, were analyzed. For breast tissues, four types of sample tissues were analyzed; they were obtained from (i) 10 normal breast, (ii) 30 benign breast lesions, including sclerosing adenosis atypical (n=10), fibrocystic disease (n=10) and simple ductal hyperplasia (n=10), (iii) from 30 pre-invasive breast lesions, including atypical columnar metaplasia (n=10), atypical ductal hyperplasia (n=10) and ductal carcinoma in situ (n=10) and (iv) from 150 invasive breast carcinomas (IBC).

### Immunohistochemistry Staining Procedure

3 micrometer thick sections were made from paraffin-embedded tissue blocks, obtained at the time of the initial diagnosis (before treatment) and fixed in AFA. Tissue sections were deparaffinized and rehydrated through a series of xylene and ethanol washes. Briefly, key steps included: (i) antigen retrieval in 0.1 M citrate buffer, pH=6 (Biocare) in a pressure cooker (4 minutes); (ii) blocking of endogenous peroxidase activity by immersing sections in 3% hydrogen peroxide in methanol for 15 minutes and subsequently rinsing them in water and PBS; (iii) incubation with primary antibodies against the targeted antigen; (iv) immunodetection with a biotin-conjugated secondary antibody formulation that recognizes rabbit and mouse immunoglobulins, followed by peroxidase-labeled streptavidin and linking with a rabbit biotinylated antibody against mouse immunoglobulin G (DAKO SA), and (v) chromogenic revelation with DAB and counterstaining with Mayer's hematoxylin. All immunostainings were processed using a Leica BOND RX research automated immunostaining device. Antibodies used were: anti-CENP-A, Cell Signaling #2186, 1/50, pH=9; anti-Ki67, DAKO M7240 clone MIB-1, 1/200, pH=9; anti-CENP-C Clinisciences #PD03 1/50, pH9; anti H3K9me3 active motif #39765 1/50, pH=9. CENP-A pattern specificity was controlled using anti CENP-A, Enzo life sciences #ADI-KAM-CC006-E 1/50, pH=9.

### Immunofluorescence Staining Procedure

For immunofluorescence analysis of breast samples and xenografts, tissues were incubated for 1 hour at 4° C. in PBS complemented with 30% sucrose for cryopreservation, embedded in tissue freezing medium (Leica) and frozen in isopentane cooled by liquid nitrogen. 20 μm cryosections were performed using a cryostat (Leica) on SuperFrost plus slides and immediately fixed with 2% paraformaldehyde in PBS for 20 minutes at room temperature. Following fixation, the tissue cryosections were incubated for 45 min in PBS supplemented with 5% fetal bovine serum and 0.3% TritonX-100 followed by incubation with the anti-CENP-A (Cell Signaling #2186, Rabbit, Polyclonal, 1/1000) in PBS 1% BSA 0.3% TritonX-100 overnight 4° C. Following 3×10 min washes in PBS supplemented with 0.1% Tween20 sections were incubated for 1 hour at RT with the Alexa fluor conjugated secondary antibody. After 3×10 min washes in PBS 0.1% Tween20, nuclei were stained by DAPI, washed 3×10 min in PBS and mounted in Vectashield on microscopic slide

### Image Acquisition and Analysis

All Immunohistochemistry staining were acquired using a scanner Philips IMS Ultra-Fast Scanner 1.6 RA. Frequency of CENP-A and Ki67 staining expressed as % of positively detected nuclei was obtained by visual examination of 100 nuclei in 50 different fields. H-score (intensity×frequency) was calculated from frequency of CENP-A positive cells and CENP-A signal intensity quantified using three staining scoring categories (1-weak, 2-moderate and 3-strong). Anisokaryosis was assessed from counterstaining as 1-mild, 2-moderate and 3-marked categories. Images were scored by 3 experienced pathologists. Immunofluorescence images were acquired using an LSM780 confocal microscope and a Zeiss Imager Z1 epifluorescence microscope piloted with Metamorph software, a ×63 oil objective lens and an ORCA-Flash4.0 LT PLUS Digital CMOS camera (Hamamatsu). Fiji software was used for Z projection with maximal intensity of Z-stacks (0.2 micron). Number of CENP-A foci were quantified with the 3D-FIED macro Cantaloube et al. (2012) from Z-stacks images acquired with the Z1 epifluorescence microscope.

### Xenografts and Irradiation

SCC61 and more radioresistant SQ20B cell lines were derived from human HNSCC (Weichselbaum, 1986 #20448). Eight to nine-weeks-old female nude NMRI mice (Janvier labs, Le Genest-Saint-Isle, France) were used throughout the study. Xenografted tumors were obtained by subcutaneous injection of 4×10⁶ SCC61 or SQ20B cells suspended in 40 μL of PBS in the mouse right thigh. Tumors were measured with a digital caliper, and tumor volumes were calculated using the following formula:

Tumor volume=length×width×width/2.

Experimental irradiations were performed using a XRAD 320 biological X rays (20 MA, 200 KV) irradiator (Precision X-Ray, Accela, North Branford, CT, USA). A fractionated 20 Gy tumor irradiation was delivered for five consecutive days (5×4 Gy) when a tumor volume of 250 to 400 mm³ was evidenced. For ethical reasons, the animals were sacrificed when tumors reached 2000 mm³.

### HNSCC Patients

The main inclusion criteria in the study were (i) diagnosis of non-metastatic locally advanced HNSCC between 2007 and 2015, according to the UICC TNM classification (Sobin et al., 2011) (ii) treatment in a curative and conservative intent by CCRT, preceded or not by induction chemotherapy, and (iii) availability of pre-treatment tumor samples collected at initial diagnosis and fixed in AFA. In total, 62 patients were included in the study. HPV status from initial diagnostic and determined Ki67 status from the original primary biopsies of the 62 HNSCC patients by immunohistochemistry staining were used in the analysis. The median age at diagnosis was 62 years (range: 56-68), 52 patients (83.9%) were males. Tumors were mainly located in the oropharynx (67.7%), the other locations were larynx (17.7%), oral cavity (8.1%) and hypopharynx (6.5%). Tumors were classified as stage IVa or IVb in 64.5%, stage III in 29%, stage II in 4.8% and stage I in 1.6% of cases. Thirty-five patients (56.5%) had HPV positive tumors. An induction chemotherapy was proposed to 24 patients (38.7%); 16 (66.7%) of them received TPF (docetaxel-cisplatine-fluorouracil), the others got either the combination of taxol-carboplatine (6 patients, 25%) or taxol-carboplatine-evelorimus through a clinical trial (2 patients, 8.3%). All patients underwent radiotherapy, 60 (96.8%) at a dose of 70 Gy and 2 (3.2%) at a dose of 74.2 or 74.4 Gy. In 42 (67.7%) cases, the radiotherapy was combined with concurrent chemotherapy or targeted therapy (either cisplatine (54.87%) or cisplatine-5FU (2.4%) or carboplatine (2.4%) or cetuximab (40.5%). At the time of data collection, the median follow-up was 7 years (range 2.3-9.6 yrs) and the median overall survival is 7.1 years (95% CI [2.5; Not reached]”.

### Statistical Analysis

Because of the exploratory nature of the study, no upfront hypothesis was made regarding the number of cases needed to achieve study's objectives; and no primary or secondary, nor any power to be reached were set. HNSCC patient characteristics are presented as mean with standard deviation (SD) when normally distributed, or as median with range (minimum and maximum) in case of skewed data. Categorical data are presented in number and proportions. Differences between continuous variables were assessed using Student's t-test or Mann-Whitney-U test, depending on normality, whereas the chi-squared test or Fisher exact test were used for categorical values. Overall survival is defined as the time between the date of diagnosis and the date of death, patients alive were censured at their date of last news. Median of follow-up and survival curves were estimated using Kaplan-Meier method and compared by log-rank test. A logistic regression model was used with the local disease control at 2 years as outcome variable and including baseline patient characteristics, HPV status, anisokaryosis, CENP-A H-score and CENP-A nuclear localization pattern (CENP-A pattern non-C or CENP-A pattern C) as covariates. Factors with a significant p-value less than 10% in the univariate analysis were included in a multivariate stepwise top-down procedure using the Akaike information criterion (AIC) and the likelihood ratio test as criteria for variable selection. The corresponding odds-ratios (OR) were calculated with their 95% confidence intervals (95% CI). A p-value less than 0.05 is considered statistically significant. All the analyses were performed using R software version 3.6.2 (R Core Team (2019). R: A language and environment for statistical computing. R Foundation for Statistical Computing, Vienna, Austria. <website: R-project.org/>).

### Results

### CENP-A Clusters in Foci at the Nuclear Periphery in Normal Human Tissues.

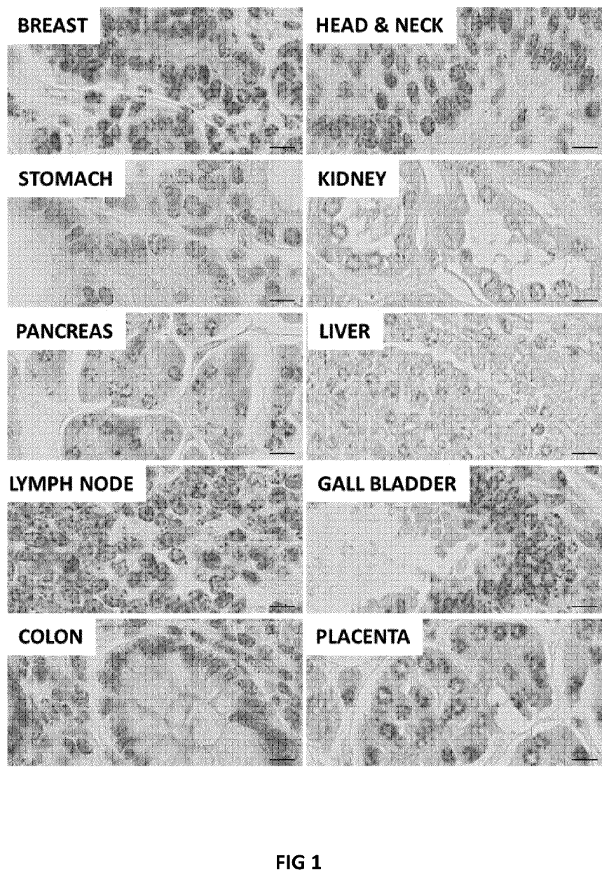

In order to investigate how CENP-A localizes in the nucleus of panels of healthy tissues, several tissues of different origin were processed for CENP-A immunohistochemistry staining. Although CENP-A staining is found to vary among tissues in terms of signal intensity (FIG. 1), in all tested tissues CENP-A is found to localize at the nuclear periphery as individual equidistant round foci of similar size, estimated in the range of 0.6 micron. In fewer cases CENP-A foci at the periphery of the nucleolus are also detected.

This localization is remarkably conserved and does not correlate with signal intensity for CENP-A. The average number of foci per IHC labeled section is 5.5 equidistant round foci per nucleus in every tissue (range is 4 to 8 foci per section). Assuming an average nuclear diameter of 5 to 10 micron depending on the tissue and 3 micron thick sections the number of foci per nucleus can be estimated in the range of 9-18 foci/nucleus. The normal human genome is composed of 46 chromosomes (22 pairs of autosomes and 2 sexual chromosomes) per nucleus in diploid cells. Therefore, 46 CENP-A foci were to be expected if all centromeres were separated and labelled. Data thus indicate that, in non-cancerous cells, several centromeric CENP-A rich regions from different chromosomes cluster together to form foci, with an estimation of 2 to 5 chromosomes in average per foci.

These data were confirmed on fresh frozen cryopreserved healthy breast tissue within which CENP-A localization was revealed by immunofluorescence staining and confocal microscopy. In these tissues, CENP-A foci were localized within the interior of the nucleus at the nuclear periphery in single focal plane. Using these 3D acquisitions, in these breast tissues, the average number of CENP-A foci detected in the nucleus is found to be 10 foci per nucleus. These data are consistent with the nuclear localization and the estimated number 9 to 18 CENP-A foci per nucleus as determined by immunohistochemistry staining on paraffin embedded tissues.

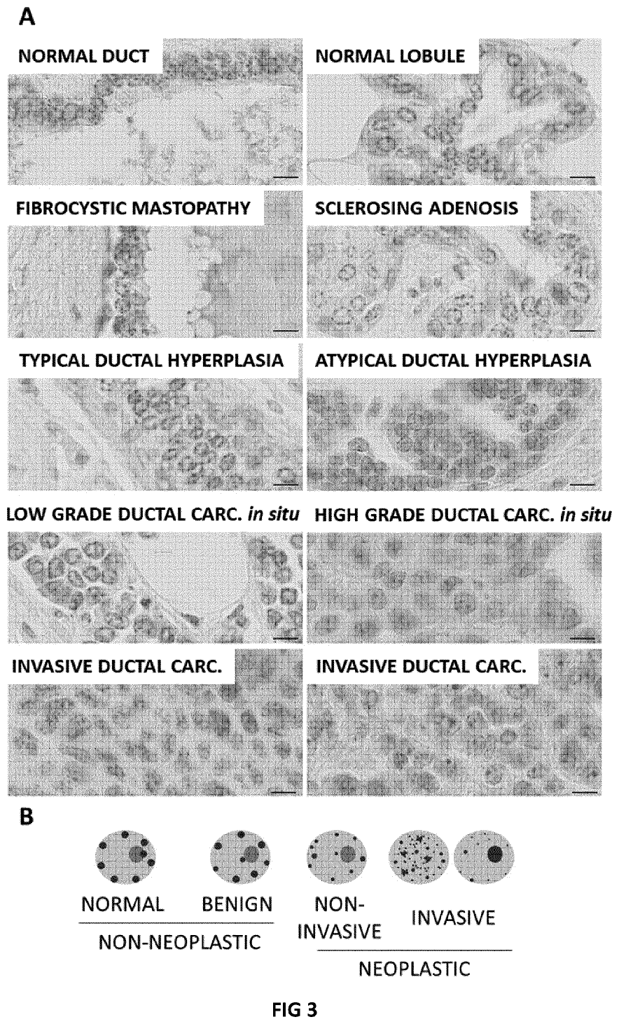

In all tissues from healthy samples analyzed, either using conventional IHC analysis or immunofluorescence staining, CENP-A localization is found to follow a well-defined pattern (FIG. 3B) characterized by:
9 to 18 individual equidistant foci that are homogenous in size and round shaped,
said foci being all positioned at the nuclear periphery within each nucleus and, in some cases, at the nucleolar periphery.

### CENP-A Nuclear Localization Undergoes Important Changes in Tumoral Tissue Samples.

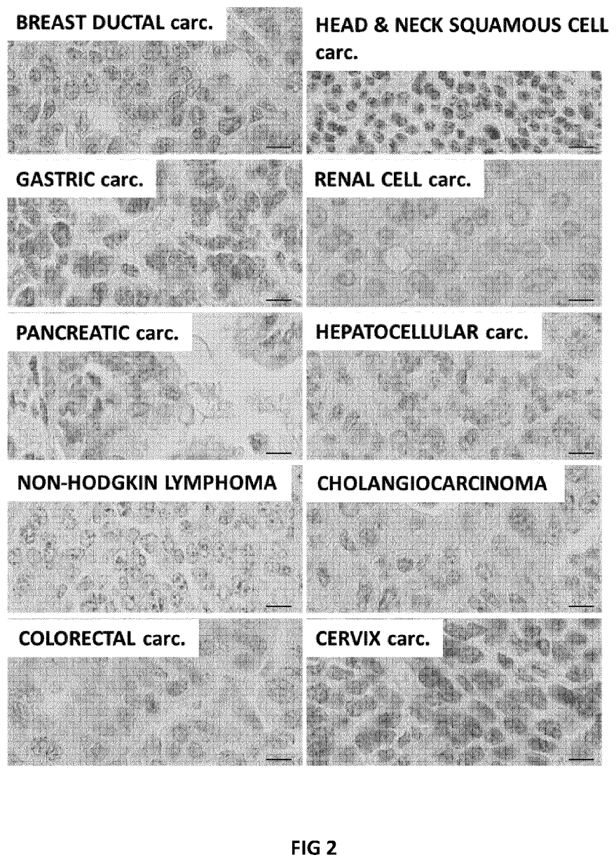

CENP-A immunohistochemistry staining on carcinomas of the same tissue origin as for the normal tissues as above (FIG. 2). In agreement with previous reports indicating a general overexpression and increased levels of CENP-A RNA and protein in tumors, an increased intensity of CENP-A staining is observed in most, but not all, tumor tissues compared to non-tumoral tissues.

Most strikingly, as exemplified in FIG. 2, CENP-A patterns in the nuclei of tumoral tissue systematically differ from that of normal tissues:

First, in contrast to normal tissue, the CENP-A foci do not localize strictly at the nuclear periphery anymore and rather localize inside the nucleus, although few foci remain at or in the vicinity of the nuclear periphery.

Second, both the round shape and equidistant localization of the foci are affected, leading to a decreased homogeneity within and among the different nuclei.

Third, the number of foci per nucleus increases concomitantly with a reduction in their size.

This alteration of nuclear CENP-A pattern was also observed through CENP-A staining and analysis by immunofluorescence on fresh frozen breast carcinomas (not shown). Compared to normal breast tissue, a decrease in the size of the individual CENP-A foci and an increased number of CENP-A foci per nucleus were observed. In addition, the number of CENP-A foci per nucleus in carcinomas is found less homogeneous, this broader range of CENP-A foci likely reflects a high tumor heterogeneity. Confocal microscopy also showed a redistribution, in regard with normal tissues, of CENP-A localization across entire nuclei: in carcinomas, CENP-A foci localization is not restricted to the nuclear periphery as in normal tissues.

CENP-A nuclear localization undergoes important changes in tumoral tissue samples which lead to a specific nuclear pattern for CENP-A labelling. Importantly, this pattern is independent of the labelling technique: Both the immunofluorescence and the immunohistochemistry data indicate that in carcinomas, the pattern of CENP-A localization strongly differs from that of normal tissue. CENP-A is not anymore detected as large foci homogeneous in size and round shaped. This likely reflects a reduced clustering of the CENP-A foci. A complete disappearance of foci could even lead to a diffuse intra nuclear staining (FIG. 2). In addition, localization of CENP-A is not restricted to the nuclear periphery anymore but rather detected in the entire nuclear space.

### CENP-A Nuclear Localization Pattern is a Marker of Cancer Malignancy

CENP-A patterns in samples corresponding to benign non-neoplastic breast lesions (dystrophic and simple hyperplastic: fibrokystic disease, sclerosing adenosis and typical hyperplasia) and neoplastic lesions of increasing malignancy ranging from atypical hyperplasia (AH) to ductal carcinoma in situ (DCIS) and invasive ductal carcinoma (IDC) were analyzed.

As shown in FIG. 3, CENP-A patterns are found strikingly different in benign and malignant lesions. Similar to what is found in normal tissues, in benign lesions, CENP-A is found to localize in the nucleus of all epithelial cells as 4-8 equidistant foci, of the same size (0.6 micron) and round shape, most often at the nuclear periphery and sometimes at the nucleolus periphery (FIG. 1 and FIG. 3). In contrast, this distinct pattern is lost in AH, DCIS and IDC. In AH and DCIS, in a majority of atypical epithelial cells, the CENP-A foci have a reduced size and sometimes are even not detected, are not localized only at the nuclear periphery and are not equidistant (FIG. 3). These changes become even more prominent in IDC, with CENP-A-foci most frequently absent from the nuclear periphery but localized inside the nuclear space. Furthermore, the number of these CENP-A foci localized inside the nuclear space is usually higher, whereas their size decreases and their shape within a single nucleus becomes heterogeneous (FIG. 3), indicative of a loss of clustering as observed above in carcinomas from different tissues (FIG. 2). Further, in the IDC samples a combined CENP-A signal is observed at the nuclear periphery, inside nuclear space and at a perinucleolar localization, leading to heterogeneous aspects among the nuclei (FIG. 3). High grade DCIS displayed a pattern similar or resembling that of IDC, which is not observed for low grade DCIS.

To sum up, features of CENP-A pattern are found to change together with oncogenic transformation and/or progression of cells (FIG. 3B):
(i) A decreased clustering of CENP-A foci (decreased size and increase of foci number) associated to progressive decrease of CENP-A localization at the nuclear periphery discriminates benign dystrophic and hyperplastic breast lesions from in situ and invasive neoplastic breast lesions (AH, DCIS and IDC),
(ii) loss of CENP-A localization at the nuclear periphery and heterogeneity in terms of number, size, and/or shape of CENP-A foci localized inside the nuclear volume within and among nuclei discriminates invasive neoplastic (IDC) from non-invasive breast lesions (AH, DCIS).

CENP-A nuclear pattern thus provide a consistent biomarker for identifying an oncogenic transformation/progression in a tissue sample, and, therefore, evaluating malignancy of the disease.

### CENP-A Nuclear Localization Pattern as a Marker of Response to Cancer Treatment

### CENP-A Nuclear Localization is Different in Radioresistant and Radiosensitive Cancers

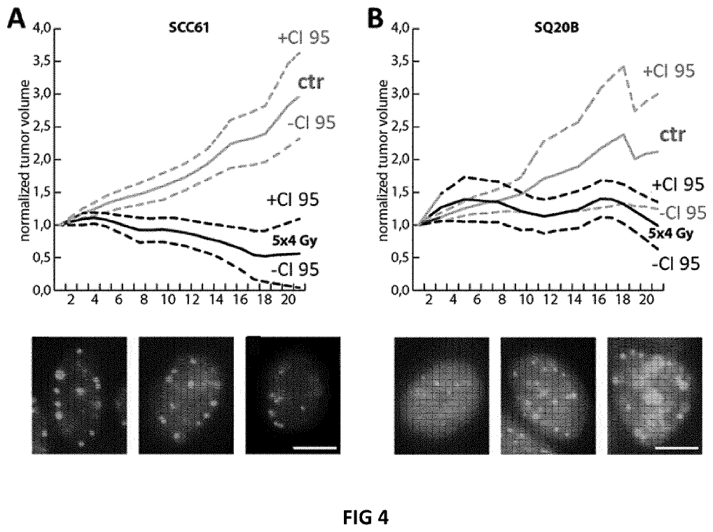

The link between CENP-A nuclear pattern and the response to cancer treatment by irradiation was evaluated using two squamous cell lines (SCC61 and SQ20B) derived from head and neck carcinomas and that are known to display radiosensitive (SCC61) and radio resistant (SQ20B) behaviors (Weichselbaum et al., 1986). In order to mimic in vivo tumor growth and because cellular three-dimensional constraints growth can modulate radioresistance (Storch et al., 2010), experiments were conducted on subcutaneous SCC61 and SQ20B xenografts into nude mice. Tumors were treated using a 5×4 Gy fractionation regimen. As expected, SCC61 derived tumors were sensitive to radiation as shown by significant decrease in size compared to control 20 days after irradiation (p<0.001, Mann-Whitney, FIG. 4). Tumor volume does not decrease in the SQ20B model upon irradiation, confirming a more resistant phenotype (p=0.2, Mann-Whitney, FIG. 4).

CENP-A localization pattern in SCC61 and SQ20B tumors prior to irradiation was analyzed by immunofluorescence staining. As observed for carcinoma-derived cells, in both cell lines, a CENP-A pattern that displayed characteristics of malignant lesions is observed, with decreased CENP-A clustering and more than 10 foci per nucleus, localization inside the nucleus and absence of systematic localization at the nuclear periphery. Remarkably, CENP-A pattern was however found distinct between the two cell lines: The radiosensitive cell line SCC61 displayed a similar intensity and homogeneous pattern within and amongst every nucleus. In contrast, CENP-A pattern in the radio resistant SQ20B cell line appeared very heterogeneous (FIG. 4). Then, in this model, different CENP-A patterns are associated with radiosensitive or radioresistant properties.

### In Human, CENP-A Nuclear Localization is Predictive of Curability by Concurrent Chemoradiation Therapy (CCRT)

CENP-A IHC staining was performed on the 62 biopsies of locally advanced head and neck squamous cell carcinoma (HNSCC) and none of them showed the pattern seen in normal tissue as described above, as expected and in line with HNSCC diagnosis.

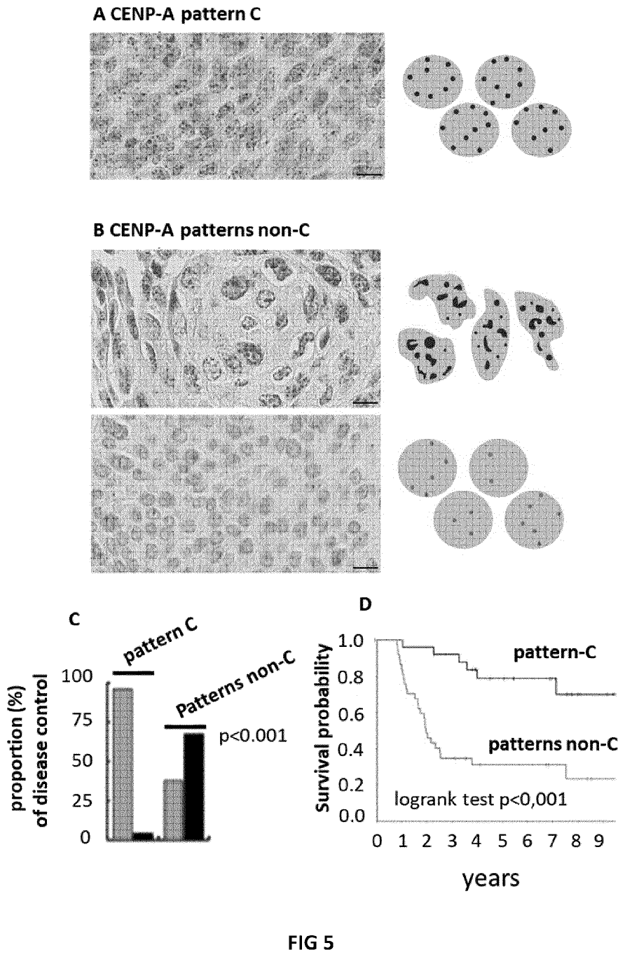

Interestingly, among the different patterns observed for the biopsies, one pattern (designed as pattern C) could be clearly distinguished and discriminated (FIG. 5A). This pattern is first characterized by its homogeneity that can be appreciated at all levels: the number, size, shape, localization and intensity of CENP-A foci appear similar for every nucleus of the section (FIG. 5A). Second, this pattern combines predominant localization inside the nuclear space and few localizations at nuclear periphery of CENP-A foci, with strong to medium CENP-A immunostaining intensity and mild anisokaryosis.

Conversely, patterns-non-C displayed heterogeneity within and amongst nuclei of the section in terms of nuclear localization, intensity, size, shape and number of CENP-A foci, with variable staining intensity and moderate to marked anisokaryosis (FIG. 4B). The pattern-C detected in the HNSCC patients is found similar to the CENP-A localization pattern of the radiosensitive cell line SCC61-derived tumor and the pattern-non-C is found similar to the CENP-A localization of the radioresistant SQ20B-derived tumor (compare FIG. 4 with FIG. 5A and FIG. 5B).

The CENP-A IHC staining of the 62 biopsies were each independently analyzed by 3 pathologists, who were asked to classify the biopsies as being of pattern-C or of pattern-non-C without any information about if the tumour was responsive (controlled) or not to CCRT. Blind analyses of biopsies showed that, in patients displaying the pattern-C for CENP-A, the disease is controlled in 96% ( 24/25) of cases, while the pattern-non-C associated with locoregional progression in 62.2% ( 23/37) of patients (p<0.001) (FIG. 5C and Table 1).

TABLE 1

| | | | CENPA Pattern | | | | |
|---|---|---|---|---|---|---|---|
| | Total | | C | | non-C | | |
| Characteristic | (N = 62) | | (N = 25) | | (N = 37) | | Test |
| Age (yr) | | | | | | | |
| Median (IQR) | 62 | (56-68) | 61 | (56-64) | 63 | (56-69) | NS |
| Ki67 (%) | | | | | | | |
| Median (IQR) | 60 | (35-75) | 60 | (35-75) | 60 | (30-75) | NS |
| Gender-N (%) | | | | | | | |
| F | 10 | (16.1%) | 6 | (24%) | 4 | (10.8%) | NS |
| M | 52 | (83.9%) | 19 | (76%) | 33 | (89.2%) | |
| T (TNM)-N (%) | | | | | | | |
| T1 | 4 | (6.5%) | 1 | (4%) | 3 | (8.1%) | 0.06 |
| T2 | 10 | (16.1%) | 4 | (16%) | 6 | (16.2%) | |
| T3 | 23 | (37.1%) | 14 | (56%) | 9 | (24.3%) | |
| T4 | 25 | (40.3%) | 6 | (24%) | 19 | (51.4%) | |
| N (TNM)-N (%) | | | | | | | |
| N0 | 17 | (27.4%) | 8 | (32%) | 9 | (24.3%) | NS |
| N1 | 7 | (11.3%) | 2 | (8%) | 5 | (13.5%) | |
| N2a | 3 | (4.8%) | 2 | (8%) | 1 | (2.7%) | |
| N2b | 9 | (14.5%) | 5 | (20%) | 4 | (10.8%) | |
| N2c | 19 | (30.6%) | 6 | (24%) | 13 | (35.1%) | |
| N3 | 7 | (11.3%) | 2 | (8%) | 5 | (13.5%) | |
| Stage | | | | | | | |
| I | 1 | (1.6%) | 1 | (4%) | 0 | (0%) | NS |
| II | 3 | (4.8%) | 0 | (0%) | 3 | (8.1%) | |
| III | 18 | (29%) | 8 | (32%) | 10 | (27%) | |
| IV | 40 | (64.5%) | 16 | (64%) | 24 | (64.9%) | |
| tumor site-N (%) | | | | | | | |
| Oral cavity | 5 | (8.1%) | 3 | (12%) | 2 | (5.4%) | NS |
| Oropharynx | 42 | (67.7%) | 17 | (68%) | 25 | (67.6%) | |
| Hypopharynx | 4 | (6.5%) | 0 | (0%) | 4 | (10.8%) | |
| Larynx | 11 | (17.7%) | 5 | (20%) | 6 | (16.2%) | |
| Metastatic | | | | | | | |
| relapse-N (%) | | | | | | | |
| No | 49 | (80.3%) | 24 | (96%) | 25 | (69.4% | 0.025 |
| Yes | 12 | (19.7%) | 1 | (4%) | 11 | (30.6%) | |
| NA | 1 | | 0 | | 1 | | |
| HPV-N (%) | | | | | | | |
| HPV− | 27 | (43.5%) | 5 | (20%) | 22 | (59.5%) | 0.002 |
| HPV+ | 35 | (56.5%) | 20 | (80%) | 15 | (40.5%) | |
| Anisocaryosis | | | | | | | |
| Mild | 28 | (45.2%) | 20 | (80%) | 8 | (21.6%) | <0.001 |
| Moderate | 19 | (30.6%) | 5 | (20%) | 14 | (37.8%) | |
| Marked | 15 | (24.2%) | 0 | (0%) | 15 | (40.5%) | |
| CENPA Hscore | | | | | | | |
| Median (IQR) | 1.6 | (1.2-2.7) | 1.8 | (1.6-2.7) | 1.5 | (0.7-1.8) | 0.003 |
| Local disease | | | | | | | |
| control at 2 yrs | | | | | | | |
| Yes | 38 | (61.3%) | 24 | (96%) | 14 | (37.8%) | <0.001 |
| No | 24 | (38.7%) | 1 | (4%) | 23 | (62.2%) | |

Correlations between patients' CENP-A nuclear pattern-(C or non-C) and response to CCRT, tumor characteristics, CENP-A amounts (H-score), proliferation (KI67), anisokaryosis and HPV status (p16) are reported in Table 1. KI67 is not correlated with response to CCRT. In contrast, as expected, 26 (68.4%) HPV positive (HPV+) patients achieved a control of their disease against only 12 (44.4%) HPV negative (HPV−) patients (p=0.02). Patients who achieved a local control displayed mild anisokaryosis in 63.2% of cases while 83.4% of relapsing patients showed moderate to marked anisokaryosis (p<0.001). Interestingly, the CENP-A pattern-C is associated with a significantly higher CENP-A H-score compared to the non-C pattern (p=0.003) and 80% of CENP-A pattern-C patients are HPV+ (p=0.002) and display mild anisokaryosis (p<0.001). Importantly, patient's age, gender, tumor stage, tumor site and Ki67 level do not differ according to the CENP-A staining pattern (Table 1).

The predictive value of those variables for local disease control using both a univariate logistic regression model and multivariate analyses (Table 2 below) for testing the probability of local disease control at two years. In both Univariate and multivariate analyses, CENP-A pattern-C stands out clearly as significantly associated and as independent predictive factor for local disease control at two years (OR=39.4, 95% CI [7.1; 744.0], p=0.001 and (OR=17.6, 95% CI [2.6; 362.8], p=0.002) respectively).

Among the controlled patients 24/38 had a CENP-A pattern-C leading to a sensitivity of the CENP-A pattern-C of 63.2% (95% CI [46; 78.2]). Among the non-controlled patients 23/24 did not have the CENP-A pattern-C corresponding to a specificity of 96% (95% CI [78.9; 99.9]) of the CENP-A pattern-non-C. Interestingly, within the subpopulation of 35 HPV+ patients, who intrinsically have a better disease control than HPV− patients, CENP-A nuclear localization pattern remains an independent predictive factors of local control at 2 years (OR=9.2, 95% CI [1.02; 203.2], p=0.048).

TABLE 2

| | | Univariate analysis | | | Multivariate analysis | | |
|---|---|---|---|---|---|---|---|
| Factors | category | OR | 95% IC | pvalue | OR | 95% IC | pvalue |
| T (TNM) | | | | NS | | | |
| | T1/T2 | 1 | | | | | |
| | T3/T4 | 0.56 | [0.1; 1.9] | | | | |
| N (TNM) | | | | NS | | | |
| | N0 | 1 | | | | | |
| | N+ | 0.57 | [0.2; 1.8] | | | | |
| Stage | | | | | | | |
| | I/II/III | 1 | | NS | | | |
| | IVa or IVb | 0.46 | [0.1; 1.4] | | | | |
| HPV status | | | | 0.02 | | | |
| | HPV− | 1 | | | | | |
| | HPV+ | 3.61 | [1.3; 11.0] | | | | |
| Anysocaryosis | | | | | | | |
| | Mild | 1 | | <0.001 | 1 | | 0.03 |
| | Moderate | 0.29 | [0.06; 1.1] | | 0.74 | [0.14; 3.9] | |
| | Marked | 0.03 | [0; 0.1] | | 0.11 | [0.01; 0.7] | |
| CENPA H score | | | | | | | |
| | | 3.1 | [1.5; 7.2] | 0.005 | | | |
| CENPA pattern | | | | 0.001 | | | 0.002 |
| | non-C | 1 | | | 1 | | |
| | C | 39.4 | [7.1; 744.0] | | 17.6 | [2.6; 362.8] | |

In line with the predictive value of the CENP-A pattern C for local disease control by CCRT, CENP-A pattern-C positive patients demonstrate a significantly better overall survival at 5 years (79%; 95% CI [64%; 97%]) compared to CENP-A pattern-non-C patients, (31%; 95% CI [19%; 51%]) (log rank test p<0.001) (FIG. 5D).

CENP-A nuclear localization pattern-C is thus a new predictive marker of local disease control at two years following CCRT, independent of HPV status and highly prognostic for overall survival.

### CONCLUSION

Experimental data provided herein do show that the CENP-A nuclear localization pattern is associated with oncogenic transformation and further, with malignancy of cancer. Moreover, inventors have surprisingly found that CENP-A nuclear localization pattern provides also a valuable biomarker for identifying predicting responsiveness of cancer to treatment.

### REFERENCES

Ahirwar R, Vellarikkal S K, Sett A, et al. Aptamer-Assisted Detection of the Altered Expression of Estrogen Receptor Alpha in Human Breast Cancer. PLOS One. 2016; 11(4):e0153001.
Cantaloube S, Romeo K, Le Baccon P, Almouzni G, Quivy J P. Characterization of chromatin domains by 3D fluorescence microscopy: An automated methodology for quantitative analysis and nuclei screening. BioEssays 2012; 34(6):509-17
Chow S, Patel H, Hedley D W. Measurement of MAP kinase activation by flow cytometry using phospho-specific antibodies to MEK and ERK: potential for pharmacodynamic monitoring of signal transduction inhibitors. Cytometry. 2001; 46(2):72-78.
Conrad R C, Giver L, Tian Y, Ellington A D. In vitro selection of nucleic acid aptamers that bind proteins. Methods Enzymol. 1996; 267:336-367.
Czernik, Production of phosphorylation state-specific antibodies. Methods In Enzymology, 201:264-283 (1991).
Ellington A D, Szostak J W. In vitro selection of RNA molecules that bind specific ligands. Nature. 1990; 346(6287):818-822.
Hofmarcher T, Brådvik G, Svedman C, Lindgren P, Jönsson B and Wilking N. Comparator Report on Cancer in Europe 2019-Disease Burden, Costs and Access to Medicines. IHE Report 2019:7, IHE: Lund, Sweden.
Huse W D, Sastry L, Iverson S A, et al. Generation of a large combinatorial library of the immunoglobulin repertoire in phage lambda. Science. 1989; 246(4935):1275-1281.
Kohman R E, Church G M. Fluorescent in situ sequencing of DNA barcoded antibodies. bioRxiv 2020.04.27.060624; doi: website://doi.org/10.1101/2020.04.27.060624
Kohler G, Milstein C. Derivation of specific antibody-producing tissue culture and tumor lines by cell fusion. Eur J Immunol. 1976; 6(7):511-519.
Lefranc M P, Pommié C, Ruiz M, et al. IMGT unique numbering for immunoglobulin and T cell receptor variable domains and Ig superfamily V-like domains. Dev Comp Immunol. 2003; 27(1):55-77.
Mahlke M A, Nechemia-Arbely Y. Guarding the Genome: CENP-A-Chromatin in Health and Cancer. Genes (Basel). 2020 Jul. 16; 11(7):810. doi: 10.3390/genes11070810.
Mullinax R L, Gross E A, Amberg J R, et al. Identification of human antibody fragment clones specific for tetanus toxoid in a bacteriophage lambda immunoexpression library. Proc Natl Acad Sci USA. 1990; 87(20):8095-8099.
Neuberger M S, Williams G T, Fox R O. Recombinant antibodies possessing novel effector functions. Nature. 1984; 312(5995):604-608.
Potts S J, Krueger J S, Landis N D, Eberhard D A, Young G D, Schmechel S C, Lange H. Evaluating tumor heterogeneity in immunohistochemistry-stained breast cancer tissue. Lab Invest. 2012 September; 92(9):1342-57.
Ramos-Vara J A, Miller M A. When tissue antigens and antibodies get along: revisiting the technical aspects of immunohistochemistry—the red, brown, and blue technique. Vet Pathol. 2014; 51(1):42-87.
Spira G, Bargellesi A, Teillaud J L, Scharff M D. The identification of monoclonal class switch variants by sib selection and an ELISA assay. J Immunol Methods. 1984; 74(2):307-315.
Sobin L H, Gospodarowicz M K, Wittekind C, eds. TNM Classification of Malignant Tumours, 7th Edition. Wiley 2011.
Steplewski Z, Spira G, Blaszczyk M, et al. Isolation and characterization of anti-monosialoganglioside monoclonal antibody 19-9 class-switch variants. Proc Natl Acad Sci USA. 1985; 82(24):8653-8657.
Storch K, Eke I, Borgmann K, Krause M, Richter C, Becker K, et al. Three-dimensional cell growth confers radioresistance by chromatin density modification. Cancer Res 2010; 70(10):3925-34.
Sun X, Clermont P L, Jiao W, Helgason C D, Gout P W, Wang Y, Qu S. Elevated expression of the centromere protein-A (CENP-A)-encoding gene as a prognostic and predictive biomarker in human cancers. Int J Cancer. 2016 Aug. 15; 139(4):899-907. doi: 10.1002/ijc.30133. Epub 2016 Apr. 21. PMID: 27062469.
Unni A M, Bondar T, Medzhitov R Intrinsic sensor of oncogenic transformation induces a signal for innate immunosurveillance. Proc Natl Acad Sci USA 2008, 105(5) 1686-1691.
Weichselbaum R R, Dahlberg W, Beckett M, Karrison T, Miller D, Clark J, et al. Radiation-resistant and repair-proficient human tumor cells may be associated with radiotherapy failure in head- and neck-cancer patients. Proc Natl Acad Sci USA 1986; 83(8):2684-8.
Zamay G S, Ivanchenko T I, Zamay T N, et al. DNA Aptamers for the Characterization of Histological Structure of Lung Adenocarcinoma. Mol Ther Nucleic Acids. 2017; 6:150-162.

### REFERENCE TO A “SEQUENCE LISTING,” A TABLE, OR A COMPUTER PROGRAM LISTING APPENDIX SUBMITTED AS AN ASCII TEXT FILE

The material in the ASCII text file, named “APIC-69158-Sequence-Listing_ST25.txt”, created Sep. 27, 2023, file size 4,096 bytes, is hereby incorporated by reference.

1. An in vitro method for identifying an oncogenic transformation in a tissue sample from a subject suspected to be afflicted with a cancer, comprising:
a step of labelling said tissue sample for CENP-A protein or for a homolog thereof, and
a step of determining a nuclear pattern of CENP-A labelling in cells from said tissue sample, said step comprising determining a presence and a subnuclear distribution of CENP-A foci in the nucleus of the labelled cells from said tissue sample.

2. The method according to claim 1 wherein the step of determining a nuclear pattern of CENP-A labelling further comprises determining, in said tissue sample:
mean number of CENP-A foci per nucleus,
size and/or shape of CENP-A foci, and/or
intra-cell or intra-tissue heterogeneity of CENP-A labelling.

3. The method according to claim 1, wherein the step of determining a nuclear pattern of CENP-A comprises detecting intranuclear CENP-A foci which are distributed only at the nuclear periphery of the nucleus, which is indicative that no oncogenic transformation occurred in said tissue sample and that said tissue sample does not display a malignant lesion.

4. The method according to claim 3, wherein from 9 to 18 CENP-A foci are detected in the nucleus at the nuclear periphery of the nucleus.

5. The method according to claim 1, wherein the step of determining a nuclear pattern of CENP-A comprises:
detecting no intranuclear CENP-A foci, or
detecting intranuclear CENP-A foci, at least a part thereof being not distributed at the nuclear periphery of the nucleus or at the nucleolar periphery of the nucleus,
which is indicative that an oncogenic transformation occurred in said tissue sample and that said tissue sample is at risk to display a malignant lesion.

6. The method according to claim 5 wherein a mean number of less than 9 or more than 18 CENP-A foci in nuclei of cells is detected in said tissue sample.

7. The method according to claim 5, wherein the step of determining a nuclear pattern of CENP-A comprises determining that at least a part of CENP-A foci are less than 0.6 μm in at least one of their dimensions.

8. The method according to claim 1, wherein the step of determining a nuclear pattern of CENP-A comprises:
detecting intranuclear CENP-A foci, at least a part thereof being not distributed at the nuclear periphery of the nucleus or at the nucleolar periphery of the nucleus, and
detecting a homogeneous intra-cell CENP-A labelling in terms of size, number, and/or labelling intensity of CENP-A foci, and
detecting a homogeneous intra-tissue CENP-A labelling in terms of size, number, and/or labelling intensity of CENP-A foci,
which is indicative that said tissue sample originates from a malignant lesion which is responsive to radiotherapy, chemotherapy and/or concurrent chemoradiation therapy.

9. The method according to claim 1, wherein intranuclear CENP-A foci are detected, at least a part thereof being not distributed at the nuclear periphery of the nucleus or at the nucleolar periphery of the nucleus, and wherein said CENP-A labelling of said tissue sample is also characterized by an intra-cell or an inter-cell heterogeneity in size, shape, or number of CENP-A foci which is indicative that said tissue sample originates from a malignant lesion which is at risk of being not responsive to radiotherapy, chemotherapy and/or concurrent chemoradiation therapy.

10. The method according to claim 1 wherein CENP-A labelling is performed using an antibody, or a fragment thereof, a monoclonal antibody, or a fragment thereof, a recombinant antibody or a fragment thereof, a nanobody or a fragment thereof, or an aptamer, directed against CENP-A.

11. The method according to claim 1 wherein said method is a chromogenic immunohistochemistry method.

12. The method according to claim 11, wherein the chromogenic immunohistochemistry method comprises a step of fixing cells of said tissue sample with a fixative containing less than 1% formaldehyde or no formaldehyde.

13. The method according to claim 1, wherein the tissue sample has been obtained from a biopsy, a fine-needle aspiration, a core biopsy, or subtotal removal of a single node.

14. The method according to claim 1, wherein the subject is selected from the group consisting of a mammal and a human subject.

15. The method according to claim 1, wherein the step of determining a nuclear pattern of CENP-A comprises detecting intranuclear CENP-A foci which are distributed only at the nuclear periphery and the nucleolar periphery of the nucleus, which is indicative that no oncogenic transformation occurred in said tissue sample and that said tissue sample does not display a malignant lesion.

16. The method according to claim 15, wherein from 9 to 18 CENP-A foci are detected in the nucleus at the nuclear periphery and the nucleolar periphery of the nucleus.

17. The method according to claim 11, wherein the chromogenic immunohistochemistry method comprises a step of fixing cells of said tissue sample with a fixative containing less than 1% formaldehyde or no formaldehyde, with an Alcohol-formalin-Acetic acid mix.

18. A method of treating a subject suffering from a cancer wherein one or more treatment options are chosen depending on a CENP-A pattern that is observed in a tissue sample from a cancer of the subject, comprising:
a step of labelling the tissue sample for CENP-A protein or for a homolog thereof;
a step of determining a nuclear pattern of CENP-A labelling in cells from the tissue sample, the step comprising determining a presence and a subnuclear distribution of CENP-A foci in the nucleus of the labelled cells from the tissue sample;
a step of choosing one or more treatment options depending on the CENP-A pattern that is observed in the the tissue sample from the cancer of the subject; and
a step of treating the subject with the one or more treatment options.

19. The method according to claim 18, wherein when the nuclear pattern of CENP-A comprises:
detecting intranuclear CENP-A foci, at least a part thereof being not distributed at the nuclear periphery of the nucleus or at the nucleolar periphery of the nucleus, and
detecting a homogeneous intra-cell CENP-A labelling in terms of size, number, and/or labelling intensity of CENP-A foci, and
detecting a homogeneous intra-tissue CENP-A labelling in terms of size, number, and/or labelling intensity of CENP-A foci, then
(a) the subject is diagnosed as suffering from a cancer responsive to chemotherapy and/or radiotherapy and/or concurrent chemoradiation therapy; and
(b) the one or more treatment options comprise a chemotherapeutic, a radiotherapeutic or a concurrent chemoradiation therapy of a tumorous lesion in the subject.

20. The method according to claim 18, wherein when the nuclear pattern of CENP-A comprises:
detecting intranuclear CENP-A foci, at least a part thereof being not distributed at the nuclear periphery of the nucleus or at the nucleolar periphery of the nucleus, and
detecting an intra-cell or an inter-cell heterogeneity in size, shape, or number of CENP-A foci, then
(a) the subject is diagnosed as suffering from a cancer likely resistant to chemotherapy and/or radiotherapy and/or concurrent chemoradiation therapy; and
(b) the one or more treatment options (i) comprise surgical resection of a tumorous lesion in the subject, and/or (ii) do not comprise a chemotherapeutic, a radiotherapeutic or a concurrent chemoradiation therapy of the tumorous lesion in the subject.
